(19) United States
(12) Patent Application Publication
(10) Pub. No.: US 2026/0109922 A1
(43) Pub. Date: Apr. 23, 2026

(54) Vertical Bioreactor Technologies Methods, Systems, and Processes

(71) Applicant: SolarClean Fuels, LLC, Fountain Hills, AZ (US)

(72) Inventor: Robert E. Falco, Fountain Hills, AZ (US)

(21) Appl. No.: 19/121,685

(22) PCT Filed: Oct. 16, 2023
(86) PCT No.: PCT/US2023/035246

§ 371 Date: Apr. 16, 2025

Related U.S. Application Data
(60) Provisional application No. 63/416,562, filed on Oct. 16, 2022.

(51) Int. Cl. C12M 1/00; C12M 1/02

(52) U.S. Cl. CPC C12M 21/02; C12M 23/22; C12M 27/00; C12M 29/04; C12M 29/06

(57) ABSTRACT

Embodiments may provide photobioreactor system such as a vertical growth enhancing mixing spectrum photobioreactor (1) including but not limited to at least one downcomer tower (22) having spiral impressions (211), an upcomer tower (21), an algae-fluid input (10), an algae downward fluid flow (25) in a downcomer tower; a gas input (18) to drive a system flow (250), a gas upward flow (20) in a upcomer tower; a carbon dioxide gas input (27) having a lower mass flow of carbon dioxide than a mass flow in an upcomer tower, a carbon dioxide gas upward flow (26) in a downcomer tower; a counterflow (230) created with the algae downward fluid flow and carbon dioxide gas upward flow; and perhaps even a collection tank (64) for dewatered algae. Each photobioreactor system may be connected to provide a plurality of photobioreactor systems of which can be automatically controlled.

This is a PCT patent application claiming priority to and the benefit of U.S. Provisional Patent Application No. 63/416,562 filed Oct. 16, 2022, hereby incorporated by reference herein in its entirety.

### TECHNICAL FIELD

Embodiments of the application relate to enhanced growth enhancing mixing spectrum (“GEMS”) mixing technologies with algae and carbon dioxide utilization. Vertical photobioreactors may be utilized to efficiently grow algae driven by air or flue gas containing carbon dioxide. Combinations of vertical and horizontal tubes may be optimal in some embodiments.

### BACKGROUND

The power generation industry is coming under increasing pressure to produce electricity from renewable energy sources. Many biofuels meet renewable energy source standards, however, sources of conventional biofuels, such as biomass, biodiesel, bioethanol, and biogas may not be uniformly distributed geographically across the nation and the world, and in general, these sources may not be located close to power generation facilities. At the same time, reductions in carbon dioxide emissions and other gas emissions from various sources are becoming increasingly necessary and desirable. In addition, the imposition, or even potential imposition, of carbon taxes could make carbon capture and utilization even more economically desirable. Typically, capturing carbon dioxide from the flue gases of anthropogenic sources, such as electric power plants, and then sequestering it may be expensive, and the long-term outcome may be uncertain (e.g., the earth may shake and may eject the gas out again, underground contamination of water supplies, and the like).

On the other hand, photosynthesis is nature's way of recycling carbon that is in the biosphere. In this process, organisms performing photosynthesis, such as plants, may synthesize carbohydrates, proteins, oils, and other cellular materials using sunlight and carbon dioxide and nutrients. One of the most efficient converters of carbon dioxide (“CO₂”) to biomass is microalgae perhaps when using solar energy in the presence of nutrients. Algae may be the fastest growing photoautotrophic organisms on earth and may even be one of nature's simplest microorganisms.

Using algal biotechnology, carbon dioxide capture can be advantageous due to the production of useful, high-value products whose productivity may be enhanced from the use of carbon dioxide that could otherwise be emitted as a waste gas into the atmosphere that contributes to global warming. Production of algal biomass as a method of reducing carbon dioxide levels in combustion gas may be an attractive concept. Algal biomass can also be turned into a high-quality liquid fuel which may be similar to crude oil through thermochemical conversion by known technologies, such as High Temperature Liquefaction (to do this economically can require supplies of waste heat and low-cost electricity), or diesel fuel (e.g., biodiesel) via the transesterification of the algal biomass's lipids, or even renewable diesel and jet fuel via modified oil refining. Algal biomass also can be used for gasification to produce highly flammable organic fuel gases suitable for use in gas-burning powerplants. Algae can produce ethanol. The protein in algal biomass and the omega-3 fatty acids can make it a good source of food, fish food and even animal feed.

Algal cultures can also be used for biological nitrogen oxide (“NOx”) removal from combustion gases. Some algae species can remove NOx over a wide range of NOx concentrations and combustion gas flow rates. Nitrous oxide (NO), a major NOx component, may be dissolved in the aqueous phase, after which it may be oxidized to nitrogen dioxide (“NO₂”) and even assimilated by the algal cell. For example, NOx removal using algae, Dunaliella, can occur under both light and dark conditions, perhaps with an efficiency of NOx removal of over about 96% (such as under light conditions).

Over an 18-year period, the U.S. Department of Energy (“DOE”) funded an extensive series of studies to develop renewable transportation fuels from algae. In Japan, government organizations, such as the Ministry of International Trade and Industry, in conjunction with private companies, have invested over $250 million into algal biotechnology. Each program took a different approach, but because of various problems addressed herein, there has been minimal large scale commercial success to date.

An additional valuable use of algae may be to produce high protein fish food that restores fatty acids to them. The need for fish farms may be rapidly increasing, and the availability of fish used to feed other fish such as salmon or the like, may be decreasing. Thus, many farms use soy and other vegetable protein for feed, but these substitutes do not contain the omega-3 fatty acids that make these farmed fish as valuable a food. Algae has these fatty acids and thus, can be a valuable fish food.

In addition, algae are capable of growing in brackish and even saline waters that may be unsuitable for agriculture. This may enable all the advantages of algae production perhaps without incurring a serious water usage problem.

A major obstacle for feasible algal carbon dioxide capture, and perhaps even pollution abatement has been the lack of an efficient, yet cost-effective, growth system. DOE's research focused on growing algae in massive open ponds as big as about 4 km². As recently as 2016, the National Algal Biofuels Technology Review Final Report (a major 3-year U.S. government funded program) concluded that, “Cultivation of algae in algal raceways and open ponds is envisioned as the most economical route for algal biomass and biofuels production. In the coming decades, if the projected scales of biofuels are to be generated, algae have to be cultured in several thousand acres of land for the desired biomass yields.” This resulted from examining many closed photobioreactor designs. Basically, the raceway ponds may require low capital input; however, algae grown in open and uncontrolled environments can result in low algal productivity primarily due to the self-shading which can occur as a result of the algae near the surface receiving sun light, growing and darkening, and creating shade over the algae in the pond further below the surface. This condition is a consequence of the fact that within a few diameters of the paddle wheel propulsion system, the turbulence it generates decays, and for the majority of the length of a raceway pond there is little mixing to bring the algae from deeper in the pond to the surface, and also to move the algae at the surface to regions deeper in the pond. An addition, perhaps due to contamination with environmental predators, slower growing algae species may contaminate the ponds. Occasionally, sudden pond death has been experienced. The open pond technology seems to have made growing and harvesting algae prohibitively expensive, perhaps because the movement of massive amounts of dilute algal waters may have required very large agitators, pumps, collectors, and the low concentration of the algae requires energy intensive drying, and the like.

To reduce the above effects, major work has been done to examine and understand the nature of biotic factors such as bacteria, viruses, invasive algal species, fungi, and even herbivores in algal ponds that may impact the algal biomass yields, and some progress has been made.

Another difficulty in using ponds to address point source discharges of carbon dioxide may be due to the shallow depth of the ponds as carbon dioxide rises to the surface and is readmitted into the atmosphere before the algae can fully utilize it.

New approaches must overcome the limitations confronting the industry today which may include the overall low area yields or even yields per unit area, which may fall far short of a theoretical maximum, and those associated with scaling up microalgal culture to commercial size. In the past, attempts have been made to scale up enclosed photobioreactors to industrial and commercial scales. As with the open raceway ponds, the key problem to solve may be the creation of mixing motions that enable all the algae to see the light entering at the walls which are needed to uniformly grow the algae in these large volume systems as the algae becomes denser. Lack of large scale mixing of dense algae in large systems blocks the light from getting to the algae in the middle of the systems (often pipes).

### DISCLOSURE OF INVENTION

The present application includes a variety of aspects, which may be selected in different combinations based upon the particular application or needs to be addressed. In various embodiments, the application may include vertical photobioreactors for algae growth, or combinations of vertical and horizontal units.

It is an object of the application to provide efficient utilization of carbon dioxide contained in flue gases by optimally growing algae in a bioreactor system.

It is another object of the application to utilize counterflow dynamics between the algal fluid and the carbon dioxide so as to engineer sufficient residence time of the gas that the algae can make maximum use of it in photobioreactor systems. A lowest energy input required to do this may be to utilize the buoyancy of the carbon dioxide that drives the gas upward and the downward flow of the algal fluid in the downcomers in a vertical photobioreactor.

It is yet another object of the application to provide algae harvesting at or near a bottom of a vertical portion of a bioreactor system.

It is an object of the application to provide a modular bioreactor system that can be scalable.

It is another object of the application to provide a bioreactor system that can be utilized on land, water, ponds, oceans, or the like.

It is yet another object of the application to provide algae to be a useful industry tool to lower carbon footprints and become a profit center.

Naturally, further objects, goals and embodiments of the application are disclosed throughout other areas of the specification, claims, and drawings.

### BRIEF DESCRIPTION OF THE DRAWINGS

FIG. 1 shows a non-limiting example of a vertical GEMS photobioreactor module in accordance with some embodiments.

FIG. 2 shows a non-limiting example of a vertical GEMS photobioreactor module in accordance with some embodiments.

FIG. 3 shows a non-limiting example of a vertical GEMS photobioreactor system having a plurality of bioreactors connected together in accordance with some embodiments.

FIG. 4 shows a non-limiting example of a flue gas algae-carbon dioxide separation and redistribution to the downcomers for sparging process in accordance with some embodiments.

FIG. 5 shows a non-limiting example of an algae harvesting method in accordance with some embodiments.

FIG. 6 shows a non-limiting example of an algae harvesting method using a Coanda screen in accordance with some embodiments.

FIG. 7 shows a non-limiting example of an algae harvesting method using a Coanda screen in accordance with some embodiments.

FIG. 8 shows a non-limiting example of a graph of algae growth comparison between a GEMS photobioreactor and an identical round tube photobioreactor in accordance with some embodiments.

FIG. 9 shows a non-limiting example of the graph in FIG. 8 with linear growth shown in accordance with some embodiments.

FIG. 10 shows a non-limiting example of growth of a high temperature algae in a 6.6 inch GEMS photobioreactor versus growth in a 5.1 inch diameter round bubble column in accordance with some embodiments.

FIG. 11 shows a non-limiting example the graph in FIG. 8 showing the effects of spiral modifications on algae growth in accordance with some embodiments.

FIG. 12 shows a non-limiting example of a photobioreactor system attached to a building in accordance with some embodiments.

FIG. 13 shows a non-limiting example of an arrangement of many photobioreactors modules in accordance with some embodiments.

FIG. 14 shows a non-limiting example of a filter separating carbon dioxide from nitrogen in accordance with some embodiments.

FIG. 15 shows a non-limiting example of a combination of horizontal and vertical GEMS piping systems in accordance with some embodiments.

FIG. 16 shows a non-limiting example of integration of a GEMS system in an ethanol-corn-fertilizer-algae industry in accordance with some embodiments.

FIG. 17 shows a non-limiting example of integration of a GEMS system in a coal or natural gas- wastewater-algae industry in accordance with some embodiments.

FIG. 18 shows a non-limiting example of photograph of an algae-carbon dioxide removal process device in accordance with some embodiments.

FIG. 19 shows a non-limiting example of photograph of an alga dewatering device depending on high streamline curvature and density difference between the algae and the water in accordance with some embodiments.

FIG. 20 shows a non-limiting example of a photograph of denser algae settling in a column in accordance with some embodiments.

FIG. 21 shows a non-limiting example of Asparagopsis algae in accordance with some embodiments.

FIG. 22a shows a non-limiting example of nodes of electrostatic waves collecting algae in accordance with some embodiments.

FIG. 22b shows a non-limiting example of nodes of electrostatic waves and the algae sinking in accordance with some embodiments.

FIG. 23 shows a non-limiting example of three outer towers arranged around a center tower in accordance with some embodiments.

FIG. 24 shows a non-limiting example of three outer towers arranged around a center tower in accordance with some embodiments.

FIG. 25 shows a non-limiting example of four outer towers arranged around a center tower in accordance with some embodiments.

FIG. 26 shows a non-limiting example of four outer towers arranged around a center tower in accordance with some embodiments.

FIG. 27 shows a non-limiting example of four outer towers arranged around a center tower in accordance with some embodiments.

FIG. 28 shows a non-limiting example of five outer towers arranged around a center tower in accordance with some embodiments.

FIG. 29 shows a non-limiting example of six outer towers arranged around a center tower in accordance with some embodiments.

FIG. 30 shows a non-limiting example of a photograph of a wire meshed reinforced vertical GEMS system in accordance with some embodiments.

FIG. 31 shows a non-limiting example of a photograph of lights used with a reinforced vertical GEMS system in accordance with some embodiments.

### MODE(S) FOR CARRYING OUT THE INVENTION

It should be understood that embodiments include a variety of aspects, which may be combined in different ways. The following descriptions are provided to list elements and describe some of the embodiments of the application. These elements are listed with initial embodiments; however, it should be understood that they may be combined in any manner and in any number to create additional embodiments. The variously described examples and preferred embodiments should not be construed to limit the embodiments of the application to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the application include technologies that can provide: scalability; driven with compressed air or even flue gas, or even nitrogen separated from flue gases; growth enhancing mixing spectrum mixing technologies; molecular filtering for carbon dioxide separation; carbon dioxide rerouting; separate introduction of carbon dioxide perhaps to produce a buoyance induced carbon dioxide counterflow; algal fluid velocities that can enable controlled gas residence time; natural algal coagulation; electrostatic standing wave coagulation enhancement; screen dewatering such as using a Coanda screen; centrifugal force via high streamline curvature algal/water separation and harvesting, phototrophic, mixotrophic, or even heterotrophic operation; automated filling, growing, and even harvesting; modularity; low capital expenditures; low operating expenses; low land area; any combination or permutation thereof; or the like.

Embodiments of the application may address the following issues: carbon utilization versus carbon sequestration; production of negative carbon dioxide organic fertilizer for improved agriculture versus chemical fertilizers; oils for renewable diesel and jet fuels; production of higher valued products; production of renewable sugars; any combination or permutation thereof; or the like.

The growth enhancing mixing spectrum (“GEMS”) mixing technologies may be used with photobioreactors such as discussed in International Publication No. WO2020/237103A1 to SolarClean Fuels, LLC hereby incorporated by reference herein. This may include tubing that allows light to pass through to algae contained therein. The tubing may have spiral impressions that can affect fluid and algae flow through the bioreactor. Such spiral impressions can be used to allow algae adequate residence time in light and dark regions as it moves in a photobioreactor.

Unlike that discussed in WO2020/237103A1, embodiments of this application provide vertical growth enhancing mixing spectrum photobioreactor systems which may include but is not limited to incorporating GEMS mixing motions, use of carbon dioxide concentration from flue gas (or other sources), routing carbon dioxide to rise counter to algae flowing down, providing counter flow-controlled residence time for maximum algae utilization of carbon dioxide, providing air or even flue gas driven reactor using low power and having low shear stresses, incorporating nano or micro bubbles in the sparging at the bottom of the downcomers, incorporating high streamline curvature for dewatering, any permutation or combination thereof, or the like.

Embodiments may include a photobioreactor system comprising at least one downcomer tower having spiral impressions configured to provide an enhanced growth mixing spectrum mixing in said downcomer tower; an upcomer tower connected to said at least one downcomer tower; an algae-fluid input configured to input a fluid having algae into said at least one downcomer tower near a top of said downcomer tower and configured to create an algae downward fluid flow in said downcomer tower; a gas input configured to input gas into said upcomer tower near a bottom of said upcomer tower and configured to create a gas upward flow in said upcomer tower to drive a system flow; a carbon dioxide gas input configured to input carbon dioxide into said at least one downcomer tower near a bottom of said downcomer tower and configured to create a carbon dioxide gas upward flow in said downcomer tower; a counterflow created with said algae downward fluid flow and said carbon dioxide gas upward flow in said downcomer tower configured to allow optimum reaction between said algae and said carbon dioxide; and perhaps even a collection tank configured to collect mature algae from said downcomer tower.

Other embodiments may include a method for using a photobioreactor comprising the steps of providing at least one downcomer tower having spiral impressions; creating an enhanced growth mixing spectrum mixing in said downcomer tower with said spiral impressions; connecting an upcomer tower to said at least one downcomer tower; inputting a fluid having algae into said at least one downcomer tower near a top of said downcomer tower with a fluid input; creating an algae downward fluid flow in said downcomer tower; inputting a gas into said upcomer tower near a bottom of said upcomer tower with a gas input; driving a system flow with said gas; creating a gas upward flow in said upcomer tower; inputting carbon dioxide into said at least one downcomer tower near a bottom of said downcomer tower with a carbon dioxide input; creating a carbon dioxide gas upward flow in said downcomer tower; creating a counterflow with said algae downward fluid flow and said carbon dioxide gas upward flow in said downcomer tower; optimally reacting said algae and said carbon dioxide in said counterflow; and perhaps even collecting mature algae from said downcomer tower in a collection tank.

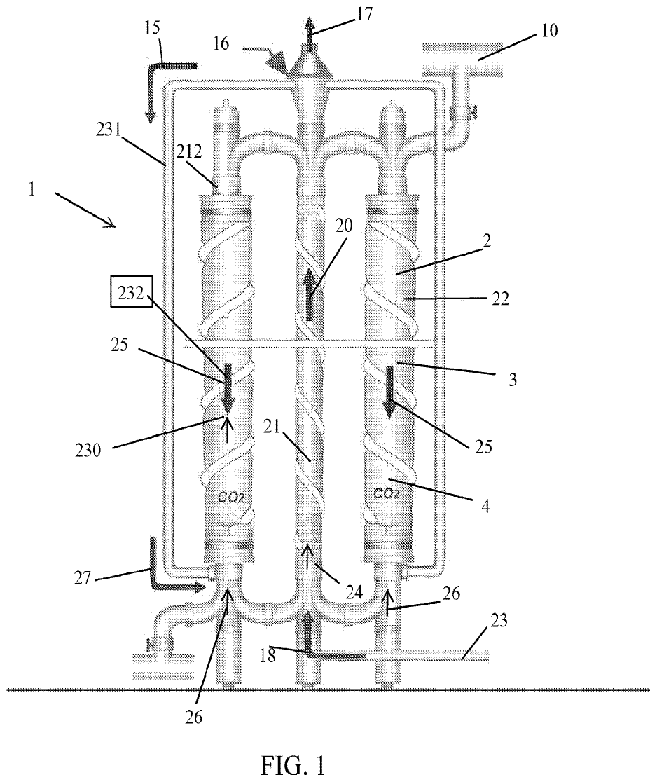

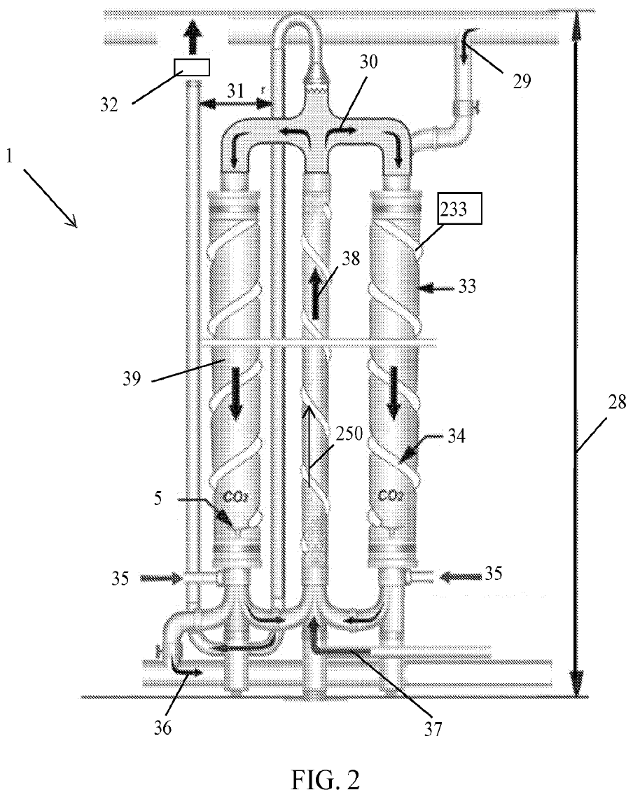

As may be understood from FIGS. 1 and 2, vertical enhanced growth enhancing mixing spectrum photobioreactor systems (“VGEMS PBR”) (1), may include three towers perhaps a middle riser tower (21) which may be called a center tower, a vertical riser or an upcomer, and two outside towers (22) which may be called downcomers or vertical downcomers. Note that any configuration (middle, outside, inside) and any number of towers may be used. A tower may be a column, a vertical tubing, structure, riser, or the like. A tower (or some or perhaps all towers and/or tubing in a system) may have spiral impressions (211) causing GEMS in fluid flow. Systems may be driven by gravity, buoyance assisted, fermentation of carbon dioxide, gas lift, air, flue gas, nitrogen, using pumps, gas compressors, or the like. A gas or gases (23) perhaps pure carbon dioxide, a gas having at least some carbon dioxide therein, or the like, may be inputted (18) into a middle riser tower and perhaps at or near a bottom (24) of the tower so that the gas can flow goes upward (20). A gas (23) may be air, a flue gas, nitrogen gas separated from a flue gas, gas from a smokestack, carbon dioxide from atmosphere, carbon dioxide gas, or the like. The gas may be utilized as a driving force, or propellant of the bioreactor. Depending on the type of gas, the towers and system may be modified for efficiency. For example, if the only available carbon dioxide is from the atmosphere, the towers may need to be taller and the sparged bubbles smaller since there may be less carbon dioxide to start, and all may be needed.

After flowing through a center riser, a gas (23) may be filtered with a filter (16) so that filtered gas (e.g., oxygen and nitrogen) can be released perhaps with a gas release (17) in the atmosphere and carbon dioxide may be circulated (15) perhaps with a gas circulation component (231) (which may be a pipe, tubing, or the like) to the outside towers (22). In some embodiments, the flue gas may not be filtered. In embodiments, the carbon dioxide gas can be circulated to a bottom (27) of a downcomer tower (22) and flow upward (26). As discussed in more detail herein, the upward flow of carbon dioxide gas can be used as a counterflow (230) to the algae moving downward in fluid downward flow (25) in the downcomer. A counterflow may allow optimum reaction between algae and carbon dioxide which may be an appropriate amount of reaction time in mixing the algae and carbon dioxide. Algae, and new nutrient loaded water may be inputted (10) at or near a top (212) of a tower. As the carbon dioxide interacts with the algae, the carbon dioxide is reduced as it is consumed by the algae. For example, at location (4) there may be about 12% of carbon dioxide in the fluid, at location (3), there may be about 6% of carbon dioxide in the fluid, and, ideally, perhaps at location (2) there may be no carbon dioxide in the fluid.

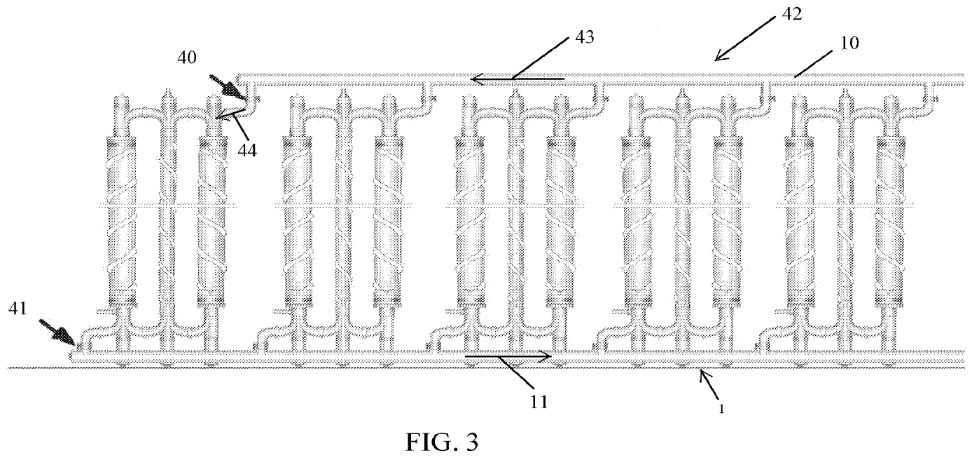

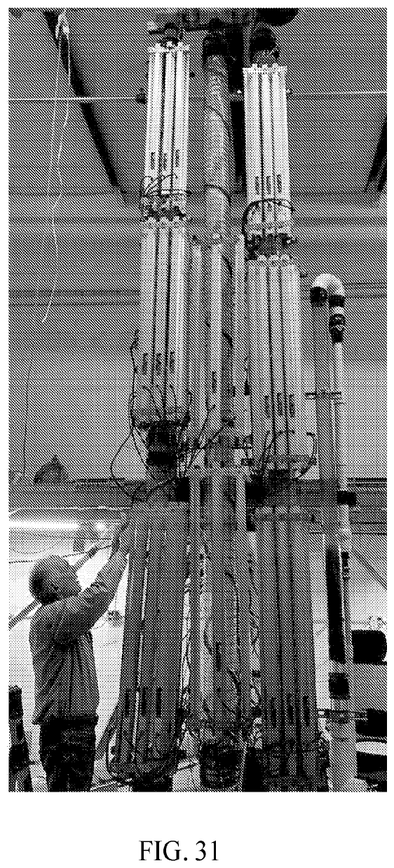

FIG. 2 provides another example of a VGEMS PBR including but not limited to: an input (29) of fluids, algae and even nutrients; clear polyethylene tubing (33) 6.6 inches in diameter has been tested (but any size can be used); GEMS spiral impressions (34) which may cause the algae to circulate in and out of light and may accelerate algae growth; a carbon dioxide input (35); a low pressure compressed air supply (37) directed up the center riser to drive the system's circulation as a system flow (250); an upward gas flow (38) which may provide a buoyancy driven recirculation (30) of algae and nutrients to the outer towers; an interaction area (39) between algae and gas; a sparger (5) which can create small gas bubbles of a size such as nanobubble size or microbubble size or the like; a gas release (32) of nitrogen, oxygen, and the like; a defoamer pipe (31) which can expel the release gas and foam (if it forms); and the like. A height (28) of a tower may vary. Towers of 17 feet high have been tested, but an optimal height will depend on the algae, the overall flow rate, the incoming gases and the sparged bubble size. FIGS. 3, 18, 30 and 31 show non-limiting examples of a constructed VGEMS PBR systems.

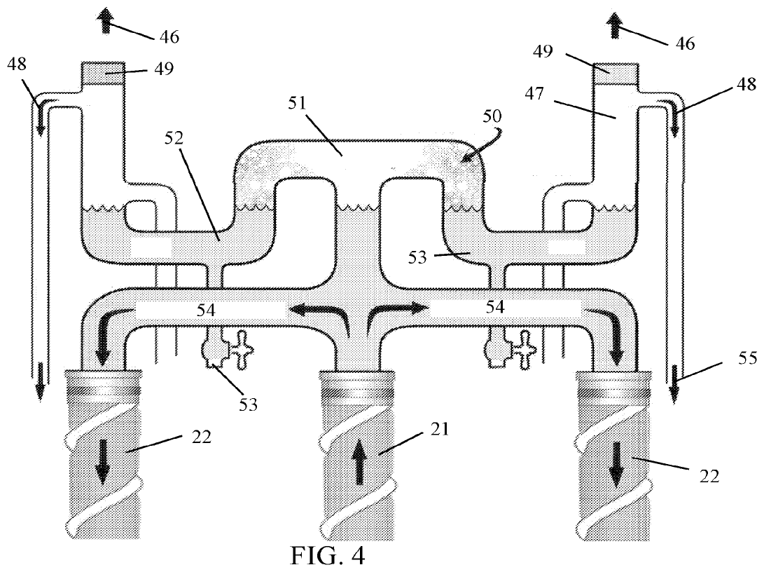

FIG. 4 shows a non-limiting example of the recirculation in a VGEMS PBR system. The top portions of a middle tower (21), and two outside towers (22) are shown. Algae that was not harvested and fluids may be recirculated (54) into the outer towers from a middle tower. A CO2 adsorbing liquid, and or a defoaming liquids (52) may be stored in a trap (53). Gases (51), such as air, flue gas, nitrogen, oxygen, trace gases, or the like may be contained in tubing at the top of a VGEMS PBR system. Here processing foam (50) may be allowed to defoam and dissolve back into a fluid. A valve (53) can be provided to clean out the liquids stored in a trap for reprocessing (if an amine) or recirculation. Carbon dioxide may be filtered with a filter (49) so that nitrogen, oxygen, trace gases, any combination thereof or the like, (46) can be released and carbon dioxide (55) can be recirculated (48) perhaps with a circulation component to a sparger, or compressor, or tank, or the like.

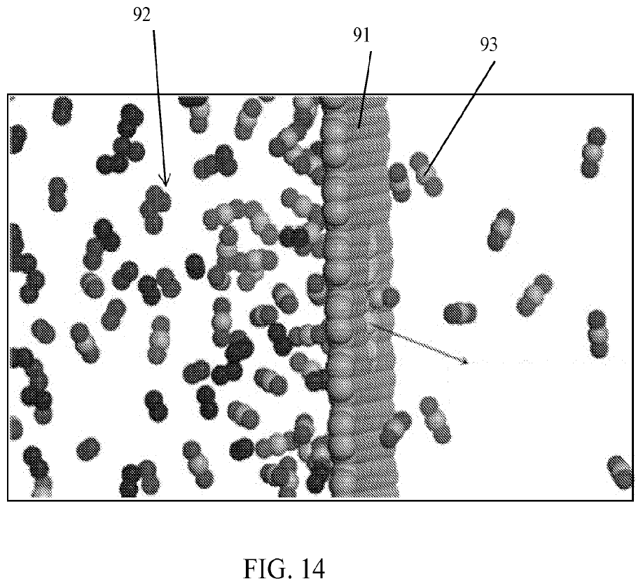

As mentioned herein, embodiments may provide that a bioreactor can be driven with flue gas perhaps from a chimney. Some flue gas may be between about 85% to about 90% nitrogen and between about 10% to 12% of carbon dioxide. Most of the oxygen in a flue gas may have been burned in the chimney. Flue gas can be inputted a photobioreactor in a center tower. In some embodiments, flue gas or air, may be used directly, in others, it may be filtered perhaps to separate nitrogen from the carbon dioxide in the flue gas since nitrogen may not be needed and may unnecessarily take up volume in the system. Certain types of algae may be able to utilize unfiltered flue gas or air such as extremophile algae. FIG. 14 shows one type of filter, a graphene filter (91), having carbon dioxide selective polymeric chains anchored on graphene which can pull carbon dioxide from a flue gas. Flue gas having a mixture of gases (92) such as nitrogen, carbon dioxide, heavy metals, oxygen, and the like, may pass through a filter to separate out carbon dioxide (93). As the flue gas passes through a filter, the nitrogen cannot pass, but the carbon dioxide can. A filter may be located at or near a top of a VGEMS PBR and even at a center riser tower. Alternatively, filters such as MOF filters, can be centrally located, and appropriated piping and pumping systems must be added to an array of VGEMS PBRs. The filtered carbon dioxide may be piped to the outside downcomer towers perhaps to a bottom of the towers so it can percolate up into the outside towers. The filtered nitrogen and any remaining oxygen can be released into the atmosphere, or collected, compressed and used as a driving gas in some of the VGEMS PBRs.

Any kind of filter, membrane, molecular filter, metal oxide framework filter, or the like can be used, they may be located anywhere in a system, and use of one or more filters may be used. In some embodiments, a single carbon dioxide filter may be used with more than one photobioreactor perhaps located centrally. In embodiments, high-performance membranes may be used. Such types of membranes can be environmentally friendly, do not generate waste, can intensify chemical processes, and can even be used in a decentralized fashion. The membranes may be based on single-layer graphene with a selective layer thinner than 20 nm and can have highly tunable chemistry. In the past membranes, may exceed 1000 gas permeation units (GPUs) and can have a carbon dioxide/nitrogen separation factor above 20 which can be a measure of its carbon capturing specificity. Newer membranes may have a six-fold higher carbon dioxide permeance at about 6,180 GPUs with a separation factor of 22.5. An increase of the GPUs perhaps up to 11,790 may be achieved when optimizing graphene porosity, pore size, and even functional groups (e.g., the chemical groups that actually react with carbon dioxide).

As mentioned, carbon dioxide or gas containing carbon dioxide may be inputted (27) at or near a bottom of each of the two outer towers into a sparger to provide an upward flow (26) of gas that is in counterflow (230) with the algae (because the algae is flowing downward and carbon dioxide may flow upward). A sparger, which may be a fine mesh disk a microbubble generator, a nanobubble generator, or the like, perhaps with a sensor may be located at the carbon dioxide input (27) where carbon dioxide can be pushed through the disk to provide fine bubbles. The carbon dioxide bubbles as a gas can rise up the tower since it may be lighter than the liquid around it and thus can create an upward flow velocity. Algae in liquid may be located in the towers and may have a downward flow down (25) in each of the outside towers. Such downward flow may work against the upward flow of carbon dioxide. The downward flow can be adjusted and can hold the carbon dioxide bubbles near a middle of a tower perhaps because the bubbles cannot rise faster than the downward fluid against them. This may be a manifestation of the counterflow. In the downcomer tower, the carbon dioxide rise velocity is being moderated by the downward flow perhaps with a downward fluid flow velocity adjuster (232) (which may be regulated by the force of the downward flow), so the algae has time to react with and consume the carbon dioxide gas bubbles. In some instances, carbon dioxide may never reach the top of the tower. This could be an optimal condition. In other instances, any remaining carbon dioxide may be reused such as by recirculated to outer GEMS towers where CO₂ may be utilized. Algae can be allowed enough residence time in the tower to use substantially all of the carbon dioxide and provide an efficient carbon dioxide utilization system. This is especially true if sparing bubble size is kept small, and may even be required to be in the nanobubble size regime. In past horizontal systems, carbon dioxide bubbles could float to the top of a pipe, may form a cloud of carbon dioxide, and be pushed along with the liquid leaving excess carbon dioxide to exhaust to the atmosphere which may not be desirable; however, the use of counterflow dynamics with carbon dioxide in a vertical photobioreactor may provide a majority of the growing algae to be exposed to the carbon dioxide counterflow for most of its life for maximum carbon dioxide utilization by the algae.

As algae flows through a GEMS photobioreactor, it may encounter dark and light phases as it moves between the middle and outside of the tower GEMS pipes. A system may include a cross-flow time scale where the algae can be made to move in between light and dark regions. This may be adjusted depending on the type of algae used, the mean flow velocity, and the depth and pitch of the GEMS spirals. Controlling the light/dark cycle by a depth and pitch of the spiral impressions and the mean flow velocity, in the presence of optimum carbon dioxide and nutrients can lead to fastest algae growth and fastest carbon dioxide utilization.

As discussed above, a sparger may be included in a VGEMS PRB system to create gas bubbles, such as carbon dioxide gas bubbles. Algae may be able to extract more carbon dioxide in a system with smaller gas bubble sizes. It may be desirable to provide a system that utilizes all or substantially all of the carbon dioxide that the algae can use over its contact time with the carbon dioxide. It may also be desirable to only introduce enough carbon dioxide into a downcomer that the algae therein can utilize. If the algae does not utilize all of the carbon dioxide, it may return to the atmosphere perhaps at the top of a VGEMS PBR tower, or recaptured and recirculated as mentioned above.

Bubble sizes may range in millimeters, microns, and nanometers. For carbon dioxide utilization by algae in downcomers, it may be desirable to use the smallest bubble size that can be consistent with complete utilization of the carbon dioxide introduced in the downcomer over the contact time with the algae. Due to a counterflow interaction between algae and carbon dioxide in the downcomer, an optimal contact time can be utilized. A bubble size can provide a bubble rise time and a counter downflow velocity can be adjusted to enable the carbon dioxide interactions with the algae. There may be an optimum velocity range for cross-flow motions in a pipe of the VGEMS PBR, thus a bubble rise velocity may need to be slightly larger than a downflow, and a bubble size may need to be optimized so that all or substantially all of its carbon dioxide is utilized before reaching the top of the downcomer. Counterflow speeds that are optimal may be a function of bubble size and even the characteristics of the algae.

In embodiments, nanobubbles may be used in the downcomers and may be introduced at a volume that can enable the GEMS motions to distribute the bubbles and carbon dioxide they carry to all the algae in the downcomer that may need carbon at a given time. In embodiments, micro or even nano bubbles may not be needed in a riser, but under certain gas mixture circumstances, they may be used.

The VGEMS PBR counterflow aspect may provide further engineering optimization potentials. For example, if smaller bubbles are used, the height of the downcomers required may be shorter and still enable complete carbon dioxide utilization by algae. This can lead to lower greenhouse costs, lower pressure at the bottom of the VGEMS tower, and even use of less robust (e.g., less costly) materials. Part of the balance may involve the higher costs of the smaller nanobubble size production devices and additional monies needed for temperature control.

Embodiments may provide other ways to increase algae growth rates and as well as optimal carbon dioxide adsorption. This may include microbubble sparging devices perhaps in the downcomer pipes as discussed herein, pulsed bubble generation and, counterflow carbon dioxide and algae flow, increasing residence time, any combination thereof, and the like.

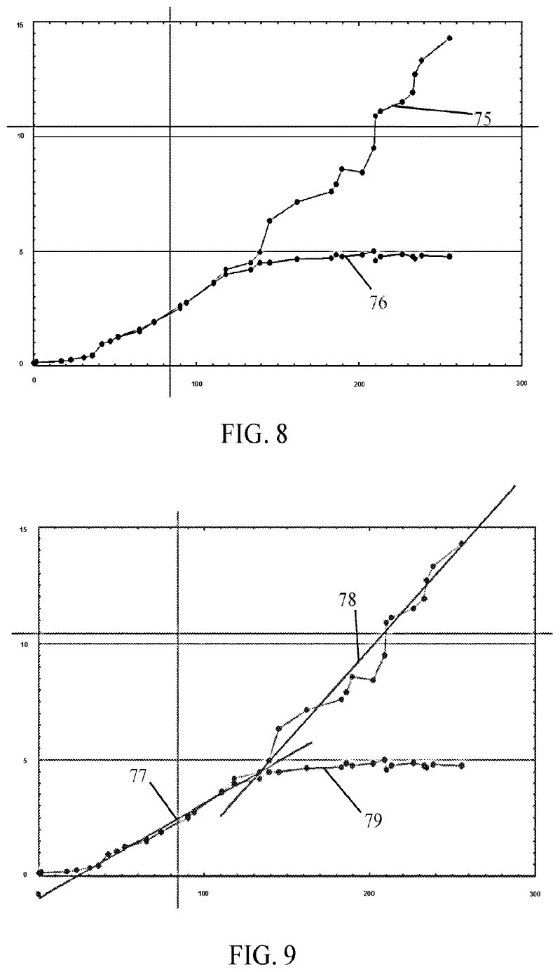

FIGS. 8-11 provides graphed results from experiments of algae growth comparison between a vertical GEMS photobioreactor and a round tube photobioreactor. The PBR was a 3 tower vertical round pipe PBR, with pipes of the same diameter, and height, but without the GEMS modifications. It was identical to the VGEMS system; had the same size, flow velocities, counterflow speeds and sparging, same upper and lower manifolds, it used the same algae, nutrients, driving air, and CO2 inputs, but it was not GEMS modified. FIG. 8 shows the growth of algae in a vertical GEMS photobioreactor (75) and the growth of algae in a round tube photobioreactor (76). As shown in FIG. 9, the results of the VGEMS PBR provided an initial linear growth rate (77). A new and better linear growth rate (78) formed later in the experiment. Here, cyanobacteria (PCC11901) which was grown in a 4.5″ diameter vertical GEMS PBR pipe, shows a faster growth rate (78) than the initial linear growth rate (77) and faster than the horizontal round pipe growth. This appears to be due to an optimization of the light-dark cycle in the vertical GEMS PBR pipe. In the round pipe, the algae growth rate slows down to zero (79) as the algae becomes dense.

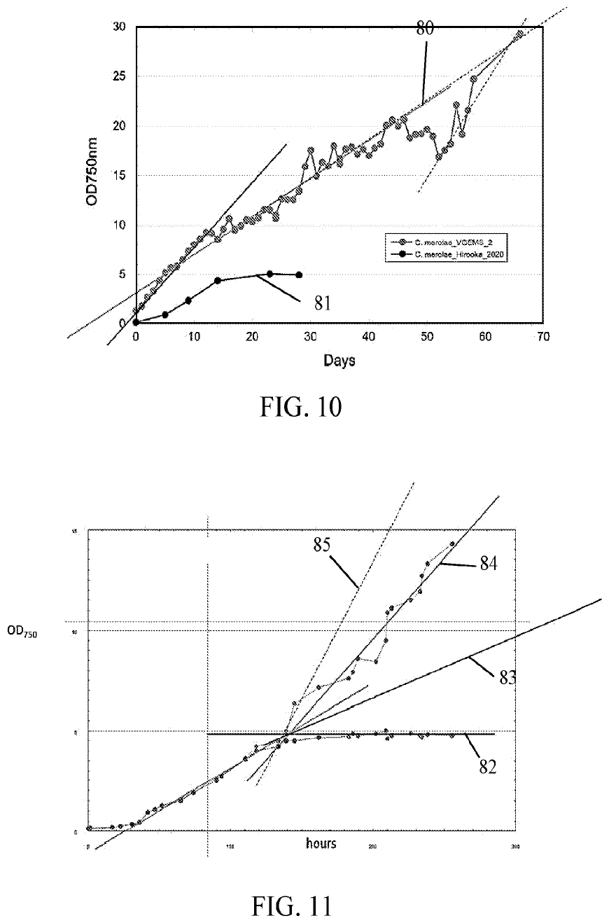

FIG. 10 provides another example using thermophilic red algae, C. merolae. Again, a second linear growth rate (80) has formed in the vertical GEMS PBR. Its growth rate is slower than an initial growth rate. However, the growth in the vertical GEMS PBR persists for almost twice the growing period as compared to the growth of algae in the round pipe (81). The vertical GEMS PBR could produce algae of twice the density to be harvested after about 60 days (note the experiment here was stopped after 48 days and then restarted). In this experiment, high temperature, C. Merolae was grown in 6.625 inch diameter VGEMS PBR pipe, and compared to data grown in a 5.1 inch diameter round pipe bubble column.

In embodiments, the light/dark cycle can be tuned perhaps for each specific type of algae to result in growth rates higher than obtained in the initial linear growth rate. This may be accomplished by adjusting the depth of the spiral impression perhaps with a spiral impression adjuster (233) and perhaps even the flow velocity for a specific algae. As such, a cross flow velocity can be adjusted and hence the time over which the algae moves across the pipe to see the light at the walls. FIG. 11 compares the different growth rates with a hypothetical faster one. The algae grown in the round pipe decreases its growth rate to zero (82) after a certain period of time. Algae grown in a pipe with a spiral impression of about 0.3 radius may have a growth rate (83) as shown. Algae grown in a pipe with a spiral impression of about 0.44 radius (depth of the spiral impression as compared to the radius of the pipe) may have a growth rate (84) as shown. A hypothetical growth rate (85) is postulated for algae grown in a pipe with a spiral impression of about 0.55 radius. As the spiral impressions get deeper, the algae growth rate seems to increase. Thus, optimization of the depth of the spiral could be advantageous in optimizing these systems.

A vertical GEMS PBR system may provide special effects that can enable better algae harvesting. As an algae flow goes down the downcomers, some coagulation may occur, and if the algal mass is heavier than water, the algae (135) will settle in a collection section at the bottom as is shown in FIG. 20. Additionally, electrostatic standing wave coagulation as shown in FIGS. 22a and 22b may be induced. Thus, the initial coagulation may be reinforced at the nodes of electrostatic waves, as shown in FIGS. 22a and 22b. Standing waves may be created by electrostatic forces. Algae may collect and may coagulate in the nodes of the standing waves.

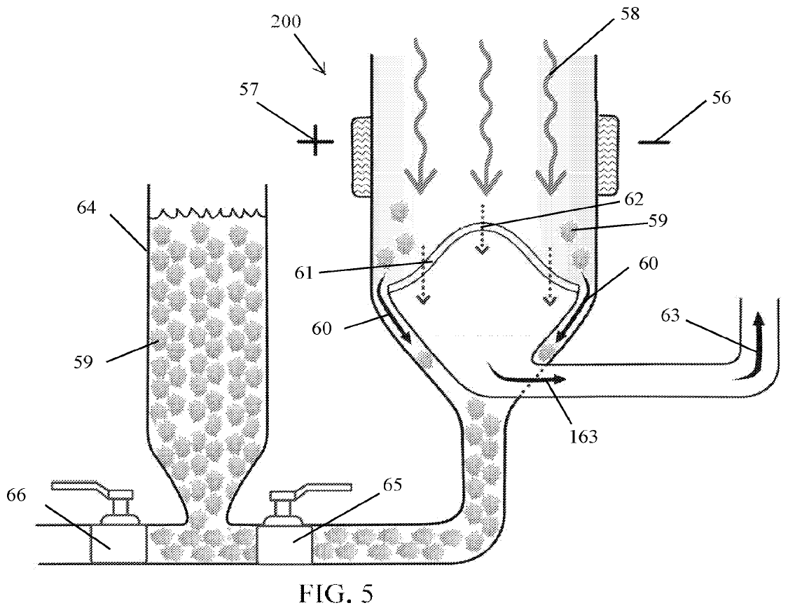

In harvesting grown algae that comes down the downcomers, a Coanda effect, gravity, and even centrifugal forces for algae and water separation may be utilized. FIG. 5 provides a non-limiting example of collection of algae near a bottom (200) of a downcomer tower. Algae and fluids may move in a downward flow (58) in a downcomer. Algae can be coagulated with an algae coagulator perhaps located near bottom of a downcomer tower utilizing a positive (57) and negative (56) charge plate connected to a system. A Coanda screen (61) may be utilized in the downcomer to cause larger mature algae and clumps (59) of algae to be directed and fall around the sides (60) of a Coanda screen and to a collection tank (64). Smaller algae and fluids may pass through (62) the screen and routed to flow (163) through a high streamlined curvature (161) in the piping where the fluids and smaller algae can be recirculated (63) through the system. Valves which may be automated may open and close to collect algae in the collection tank. For example, valve (65) may close and valve (66) may open to harvest the collected algae (64) from the collection tank (64).

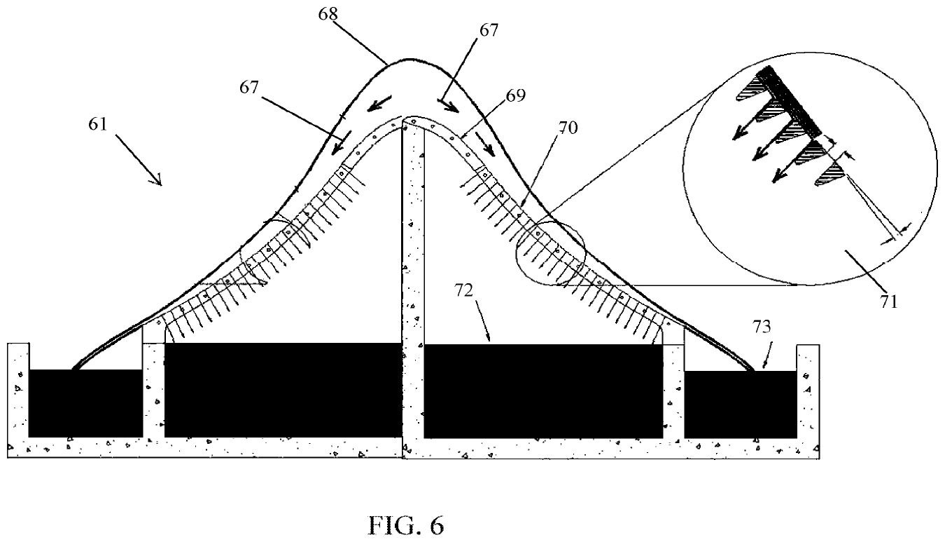

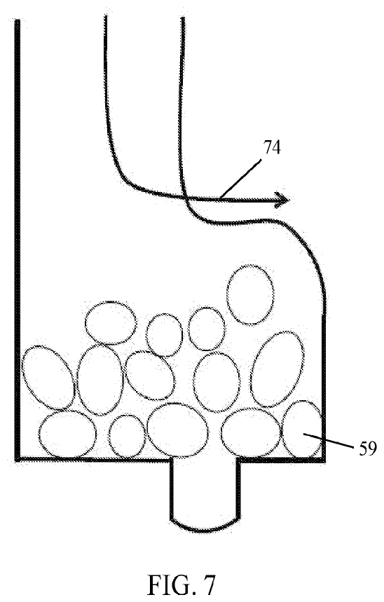

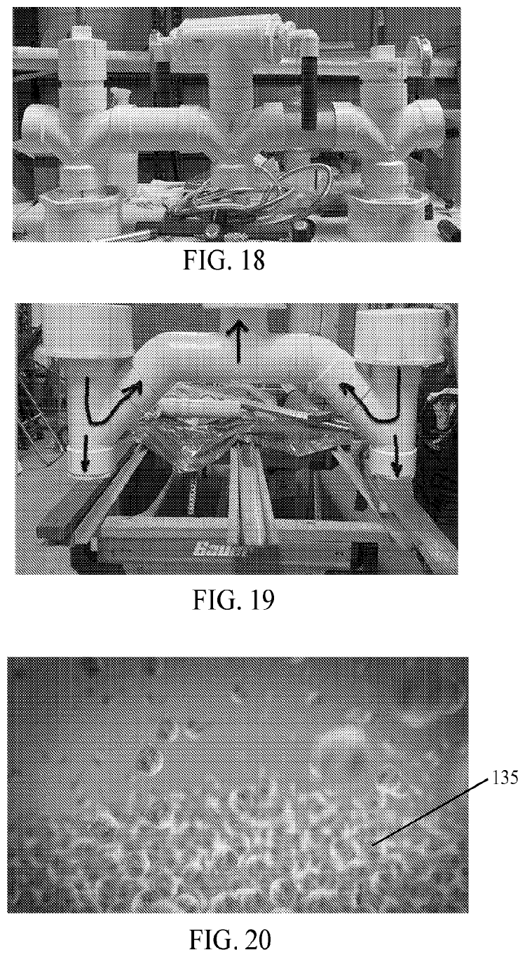

More specifically, algae can be harvested using curved streamlines of a VGEMS PBR system. The inertia of a heavier, larger, more mature algae particle could keep it moving its current direction such as in a straight line or a straighter line than the streamlines made in an overall VGEMS PBR geometry. Other less dense particles, such as lighter algae, fluids, and nutrients may flow along the streamline curve where they can be recirculated in the system. Gravity and a high streamline curvature (161) in a pipe may assist in continual algae harvesting. Algae may be more dense than water so that when a steam flow of a mix of algae and water reaches a high streamline curvature in a pipe, algae and heavier algae may not follow the fluid direction (74) of a streamline curvature and may fall into a bottom of a tower whereas the fluid may follow the curvature and allow separation of the algal particles as shown in FIG. 7. FIG. 19 provides a photograph showing a non-limiting example of high curvature in the piping of a vertical GEMS PBR system that can provide a high curvature flow (74) and enable harvesting of grown algae. Here a non-limiting example of PVC pipe is used to provide a curved streamline effect. The geometry is such that the algae goes below the level needed to make the turn to go into the riser, and is then forced upward to continue on its circulation path to the riser. This manifold was built from off-the-shelf 6″ diameter PVC parts and has a 135 degree bend at the elbow joints. A liquid flow may come down the two outer downcomer tubes, may be sped up by an area contraction, and then may go up the center riser to complete the circuit. Algae in the flow, would have to go around the 135 degree bends of the streamline curvature. A system may be designed with any size of piping and any shape bend to optimize harvesting of the grown algae. The inner radius of the turns can be adjusted to reduce the shearing effects. The algae that cannot make the turns can fall into the collection zones (see arrows).

It may be desirable optimize the flow forces so that the mature, heavier algae do not make the turn and thus move down into a collection area. The parameters may involve a combination of the mean flow velocity of the downcomer and the radial velocity of the mean flow streamlines in the return bend created by the curvature of the streamlines in the bend. Systems can be custom made perhaps with custom molds and as such, the radius of curvature of the bends can be customized. There may be a large range of flow velocities to evaluate in a creating a customized system for use with different types of algae including but not limited to micro bubble sizes, counterflow dynamics, degree of bend, freedom of area ratio in a bend, and the like. The instantaneous velocities may be responsible for the ultimate trajectory of any algal cell, thus it may take a number of circuits around the VGEMS PBR before an algal cell, even a mature cell, can fall into a collection zone and is harvested. The ultimate custom configuration may be determined by trial and error, but the same concept can be embodied in all the subsequent configurations.

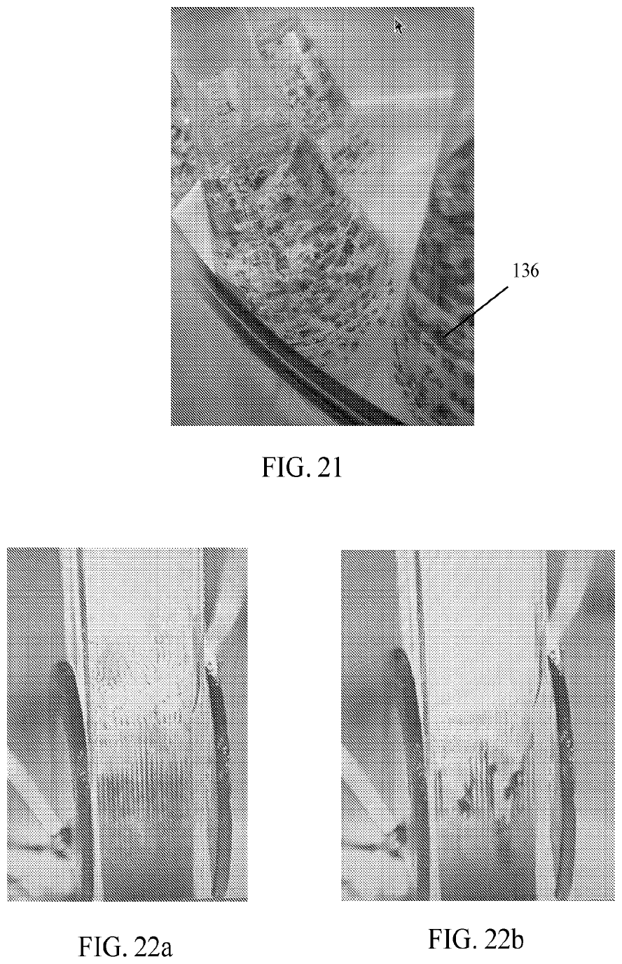

Depending on cell wall toughness, density differences, and perhaps even the size of algae cells, separation of fluid and water from algae cells may be achieved. An algae filter and dewaterer may be configured to filter and remove water from mature algae perhaps using the Coanda effect. FIG. 6 shows an arrangement of a Coanda screen (61). A Coanda screen or other type of filter may be a bell curve shape which may direct fluid flow (67) from an upstream pool (68). It may include an acceleration plate (69) and wedge-wire screen (70) perhaps like teeth. A Coanda effect (71) may keep a flow attached to a top surface of each wire screen. Tilted wires can shear flow through the screen. A diverted flow (72) and a bypass flow (73) is shown. When a fluid flow and algae passes the screen, the fluid may flow by and even around the corners and back into the bioreactor but the coagulated algae may be collected near the bottom for harvesting. A screen can allow small algae through the screen with the water (perhaps for further growing) but can deflect the larger clumps of algae around the sides of it for collection in a collection tank. Smaller algae can move within a tower and may be recirculated. Without a screen, all of the algae in a tower may continue to recirculate and it may be difficult to concentrate for harvesting. With a filter or screen, the bigger algae clumps may be separated. Collection of algae may be easier with a screen perhaps because the algae may not have as much water with it. In embodiments, Asparagopsis algae (136), shown in FIG. 21, can be separated with a Coanda screen. Of course, combinations of physical effects can be utilized to optimize dewatering and harvesting, for example sequential systems of Coanda screens and high curvature centrifugal dewatering may compliment each other.

Systems may be modular and may be scalable. Modularity may enable organic growth of a bioreactor system. Because of the large number of modules, maintenance of any module in the system may effectively not interrupt an overall operation. Scalability may enable a bioreactor system to meet individual demands that could be large scale or even small scale.

FIG. 3 show a non-limiting example of a vertical GEMS photobioreactor system having a plurality of VGEMS PBRs (1) connected together. These figures show a schematic of piping which can be used for automatic refill and emptying of the connected VGEMS photobioreactors (42). An input (10) of material may flow (43) into a system perhaps via a single pipe or multiple pipes. This input may include fluids, algae, algae stock, nutrients, any combination thereof, or the like. Such input may then flow (44) into each VGEMS PBR unit, here 5 units are shown in FIG. 3, however any number of reactors may be combined. Each bioreactor may be separately controlled using separate operation controls, that may be automatic, may be in response to sensor inputs from each separate system and may control a valve input (40) and a valve output (41) with a plurality of valves in each system. When ready, mature algae and fluids may be outputted from each bioreactor perhaps via an output (11). (The aforementioned Coanda and high curvature dewatering equipment geometries are not shown in the schematic.) Then, a new input of fluids, algae stock, nutrients, or the like can be refilled and inputted into each bioreactor, algae may be generated, and when ready algae and fluids may be outputted again. Some algae may remain in the system perhaps to seed new algae production. This cycle can be refilled and emptied as many times as needed. As may be governed by sensors, the operation can be completely computer controlled. The sensors may indicate that one or more modules are ready to be harvested at any given time, although the figures may indicate the 5 modules shown are being harvested simultaneously. In embodiments, parts of or even the entire system may be automated.

Systems can be scaled up by making the pipes bigger and replicating to a large system. In some embodiments, modular systems can be organically increased as needed. For example, a user can start with 5 VGEMS PBRs, then as the company grows, about 5 more can be added, and the like. Each VGEMS PBR can be a different size, such as using larger pipes, longer pipes, smaller pipes, shorter pipes, and the like. As a VGEMS PBRs system gets bigger, the overall capital expenditure may become less especially using the flexible plastics, and further due to less valves needed, less leaks, and the like for a given volume.

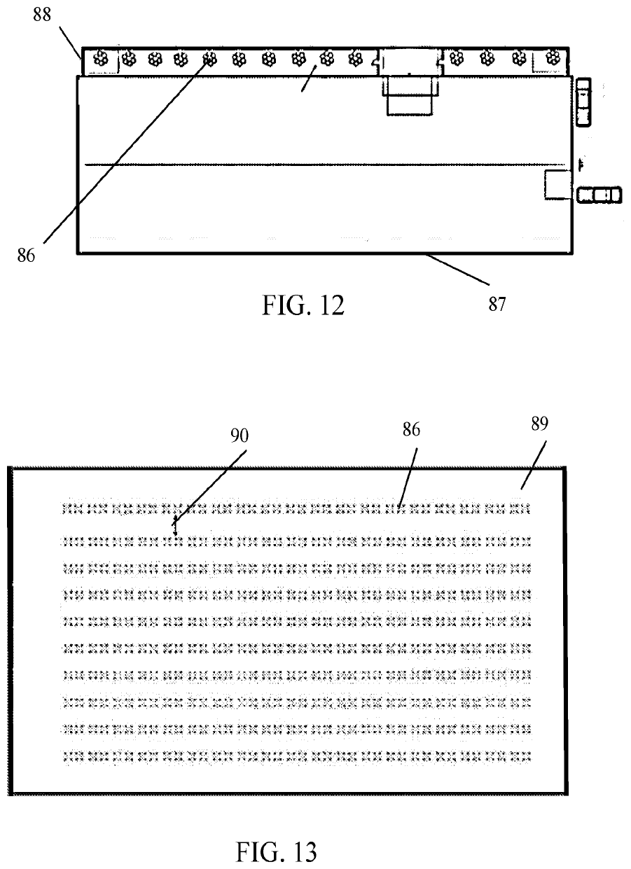

VGEMS PBR systems can be added onto existing buildings (87) as shown in the non-limiting example of FIG. 12. A new system (88) may be attached to a side of a building perhaps in a single layer of connected VGEMS PBRs (86). Each bioreactor may be connected to form a configuration. For example, a stack may have 6 VGEMS PBRs; however, any configuration may be used with any number of bioreactors. The type of system may be based on the type of algae, the amount of carbon dioxide available, the amount of wastewater available, the lighting arrangement, and the like. The side of the building with the VGEMS PBRs may be a southern side to take advantage of the sunlight. The modularity of systems can allow versatility in use to accommodate the user and their needs. FIG. 13 shows a non-limiting example of an arrangement of 380 VGEMS PBR modules of 4 downcomers and one riser. Depending on the size of the modules and systems, this could be placed in a half acre building or the like. Each VGEMS PBR (86) may be arranged (89) in a building. They may be organized in rows and columns and rows could be a certain distance apart (90) such as about six feet apart for fire and maintenance.

As mentioned herein, embodiments of the application may include systems with valves (such as solenoid valves) which may be automatically operated with a computer program perhaps to open and close the valves. Valves may be included in each VGEMS PBR module, each tower of each module, and connected in an array of modules. The valves can help fill and empty the reactors whether it is for one VGEMS PBR, or a plurality of about 5 to about 500 or more reactors. Valves, which may be computer controlled, may be located a top of a vertical bioreactor system and perhaps another valve at a bottom. A pressurized feeder line input may feed nutrients and perhaps algae stock, etc. into a system as needed. An output line may be a suction line to draw out algae when mature and even ready to be harvested. A bioreactor can be emptied individually, or if attached to others, could be emptied together or a system can pick and choose when to empty what bioreactor at different times. An input which may be a pipe may be under pressure and filled with nutrients but in some instances cannot be inputted into a bioreactor until a valve may be opened. To fill an empty reactor, a top valve may be opened and a bottom valve may be closed. There may be some gases that need to be released. Once a VGEMS PBR may be full, the algae may circulate and grow and perhaps after an amount of time (e.g., about 5 days, about 10 days, this can be any time perhaps based on the type of algae), it can be emptied. Once emptied, the VGEMS PBR may be cleaned and refilled to begin a new cycle. Sensors in a system may provide data so that the system can automatically determine when bioreactors need to be filled, emptied, etc. Furthermore, embodiments may provide for an automated system where harvesting and intermittent refilling of the bioreactors may be continuous.

Embodiments may provide VGEMS PBR systems that can be located on land, in the ocean, on water, bay, cooling ponds of power plants, and the like. In water, such as in the ocean, systems may be tethered to a structure such as a windmill, sea floor, or the like. In the ocean, the temperature of the ocean may be used to moderate the temperature of the VGEMS PBRs. In some embodiments, about a third (or more or less) of a portion of the bioreactors may float above the sea line and be exposed to sunlight (this can be adjusted using floats). In some situations, a mixotrophic algae may be used which can grow in both dark and light and can be mixed in between the light and dark areas of reactors. VGEMS PBR systems may be located next to a coal fired power plant, a wastewater treatment plant, and the like and can utilize the carbon dioxide waste and/or wastewater from them as discussed in more detail herein. In embodiments, bioreactors could be floating in water or in a cooling pond perhaps to avoid taking up land space. Such system may be closed loop and may avoid additional building costs.

Embodiments may provide an increased algae generation per unit of acre perhaps due to the vertical nature of some systems. Traditional algae growth systems that occur in a pond may be solely horizontal systems. Some algae can live on very little carbon dioxide and can survive for a long time in which carbon dioxide from the air could be sufficient to support a system. Such systems may be effective in direct air capture systems or may be used in combination with other direct air capture systems that can concentrate carbon dioxide.

In embodiments, a photobioreactor system may include a combination of vertical and horizontal piping systems such as shown in a top view of a system in FIG. 15. Horizontal systems may be understood in WO2020/237103A1. Vertical risers and downcomers or a combination of horizontal GEMS pipes and vertical GEMS pipes may be used in various systems. FIG. 15 shows four VGEMS PBR modules (95) connected to horizontal piping (94). By including horizontal PBR in a system, it may result in a lower cost as long as there is enough space to include the horizontal PBR sections. The horizontal sections of this system may need one of many possible ways to enable gases and fluids to move (96) along the pipes. This may be accomplished by attaching short sections of spiral with spacers, short sections using various connectors that enable gas passage, squashing the spiral in places, or the like so as to allow a gas passage.

In embodiments, direct air capture may utilize a filter to separate carbon dioxide from the atmospheric air, concentrate it and the concentrated carbon dioxide may be used to grow algae faster. In this type of application, perhaps 4 to 6 downcomers may be used per riser though any configuration may be used. Maximization of mass of counterflow may be desirable. A riser used for this purpose may not need to be a GEMS riser, as compressed air would be the driving gas, but the GEMS could be used.

The risers, downcomers, piping thereof, and tubing may be made of a plastic material. Continual examination of break strength and tensile strength and clarity of catalytic mixtures of LLDPE and metallocene and other catalysts may be needed to improve the combination of the material used in the piping. Construction thereof may include methods for laying of the spiral; possible correlation of the slope of the ground with depth of the spiral perhaps to facilitate gas passage; and if the application requires rigid tubing, a spiral groove may be impressed during pipe's extrusion with ball bearing race, bolted onto the extruding machine; and the like. For soft plastics, a mesh covering may be used which may extends the range of plastics that can be used. Metallocene catalyzed Linear Low Density Polyethylene's offer a wide range of properties, as a non-limiting example. The right combination of flexible plastic tensile strength, secant modulus, mesh size, mesh material, mesh material strength, and VGEMS liquid height, can enable inexpensive plastics (with respect to rigid pipes) to be used in many cases.

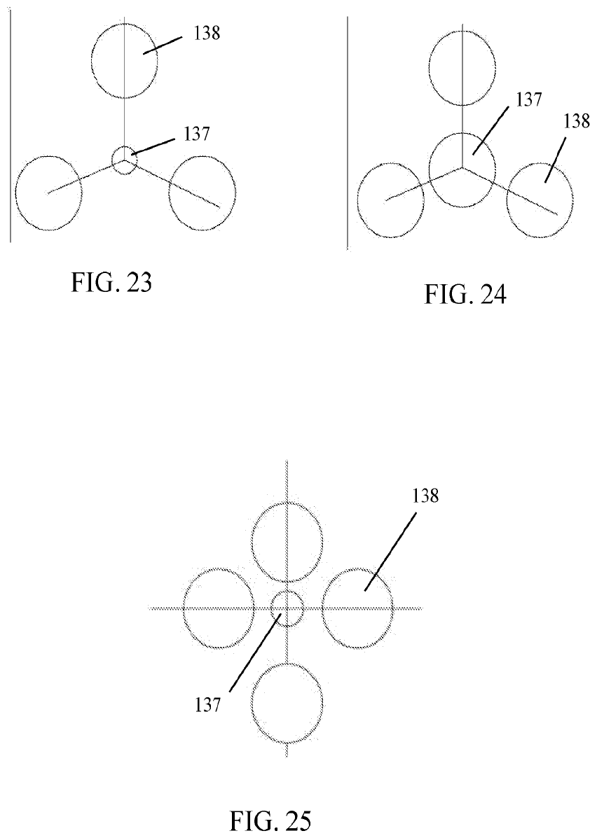

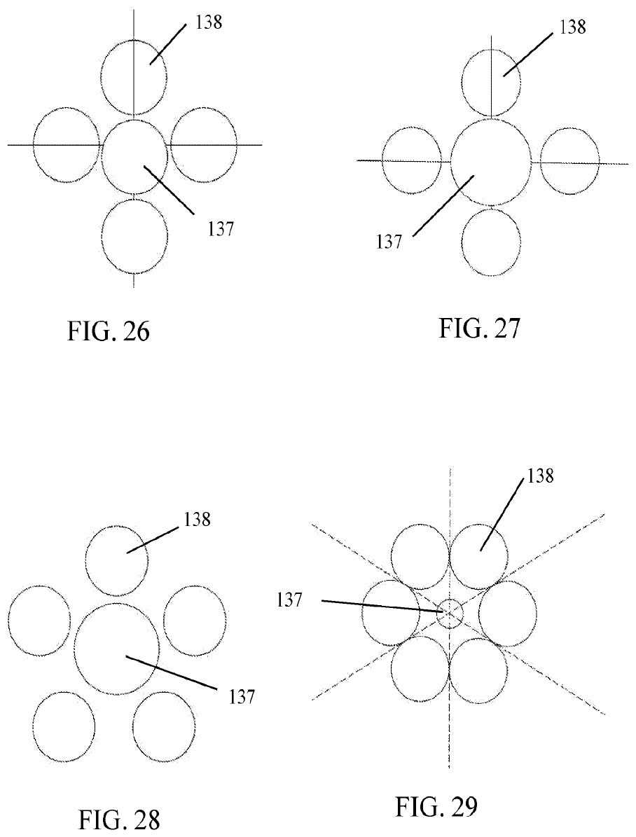

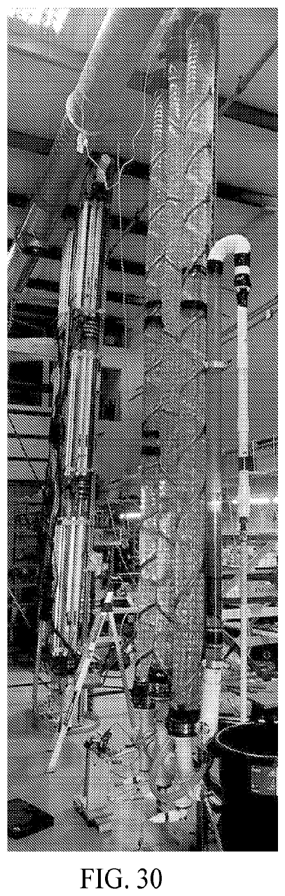

In embodiments, systems can be designed based on the algae used and even the industrial circumstances. A bioreactor system and even configuration thereof can be constructed to accommodate the rate of effluent flow needed to be handled, and even accommodate the speeds at which the algae can be moved without damaging it due to the higher speeds in the center riser tower. For example, the larger diameter the center tower, the slower the algae can move in the riser while still having the necessary velocity in the surrounding outside towers which may be downcomers, to optimize CO₂ uptake by the algae. Therefore, in embodiments, a vertical bioreactor system can take on any different configuration, including but not limited to one, two, three, four, five, six, seven, eight, nine, ten or more downcomer towers perhaps with a single riser tower as may be understood from FIGS. 23-29. This may be determined by the type of algae used and the application utilized. A center riser tower (137), can be of smaller or even larger diameters for any configuration of downcomers (138) which may be dependent on the energy inputs available and perhaps even the speeds the algae can handle.

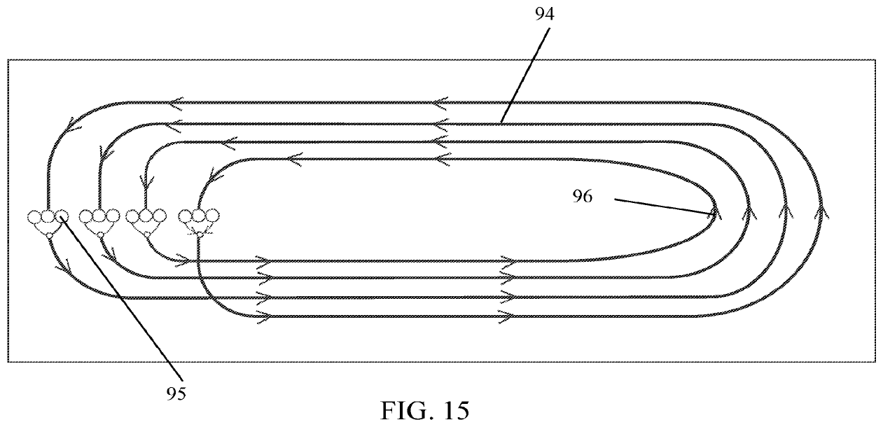

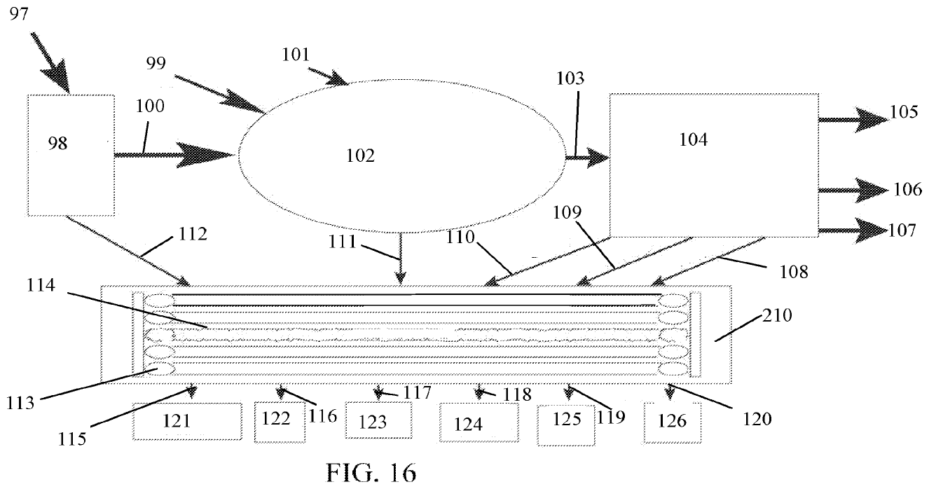

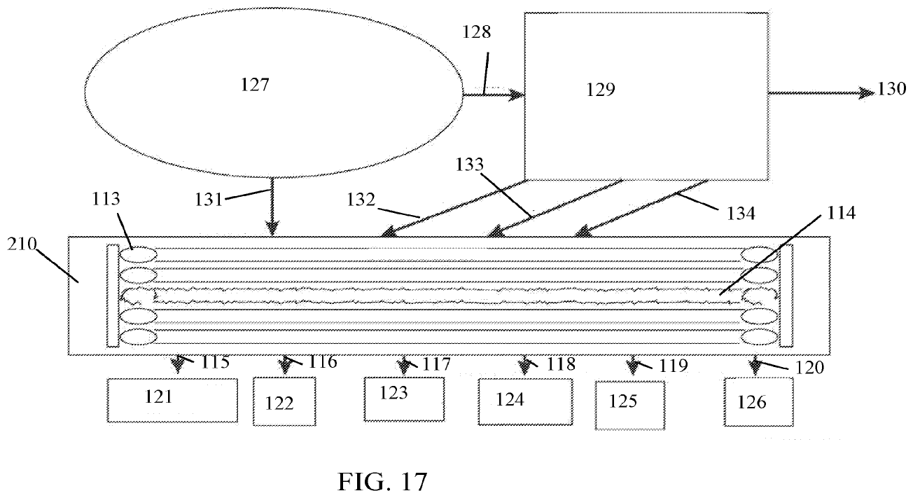

Embodiments may include integration of a VGEMS PBR system with other industries. FIG. 16 shows an integrated ethanol-corn-fertilizer-algae system and FIG. 17 shows an integrated coal or natural gas-wastewater-algae system. In FIG. 16, corn fields (102) can be used to grow for food and fuel and may utilize carbon dioxide (99) and water (101). Of course, other crops may be substituted. A fertilizer plant (98) may convert methane (97) into ammonia and nitrogen (100) which can be by the corn field and such reaction may release carbon dioxide (112) from the plant. These carbon dioxide emissions (112) can be supplied to an algae photobioreactor system (210). Corn (103) can be harvested from the field and may be processed at an ethanol plant (104) to provide ethanol (105), cow feed (106), and even (107) renewal jet fuel. An ethanol plant may also create carbon dioxide emissions (110), thin stillage (109), and even waste heat (108) which may be inputted into an algae photobioreactor system (210). The fertilizer used in the corn fields can provide a high ammonium runoff (111) of which can be supplied to algae in the photobioreactor system (210) to help their growth. An algae photobioreactor system (210) may include VGEMS PBR (113), horizontal photobioreactors (114) with and without GEMS, and any combination or permutation thereof. A photobioreactor system (210) may produce: oil (115) which can be used in renewable jet sustainable aviation fuel (121); nutraceuticals (116) which can be used in omega 3 and omega 6 supplements (122); organic fertilizer (117) which can be used with organic produce (123); astaxanthin (118) which can be used with human antioxidants (124); protein (119) which can be used for cattle, fish, and the like (125); and perhaps even phycocyanin (120) which can be used for humans and food (126).

In FIG. 17, a wastewater treatment plant (127) may provide water (128) that can be supplied to a coal or natural gas powerplant (129) to create electricity (130). A wastewater treatment plant may create an effluent (131) with phosphates and nitrates that can be supplied to a photobioreactor system (210). Carbon dioxide (132), electricity (133), and perhaps even waste heat (134) may be generated by the power plant and be supplied to the photobioreactor system (210). As discussed in FIG. 16, a photobioreactor system may utilize algae with PBRs to product many reuseable products while utilizing carbon dioxide emissions and other waste products from commercial processes to fuel the photobioreactor system.

While the invention has been described in connection with some embodiments, it is not intended to limit the scope of the application to the particular form set forth, but on the contrary, it is intended to cover such alternatives, modifications, and equivalents as may be included within the spirit and scope of the application. Examples of alternative claims may include:
1. A photobioreactor system comprising:
   at least one downcomer tower having spiral impressions configured to provide an enhanced growth mixing spectrum mixing in said downcomer tower;
   an upcomer tower connected to said at least one downcomer tower;
   an algae-fluid input configured to input a fluid having algae into said at least one downcomer tower near a top of said downcomer tower and configured to create an algae downward fluid flow in said downcomer tower;
   a gas input configured to input gas into said upcomer tower near a bottom of said upcomer tower and configured to create a gas upward flow in said upcomer tower to drive a system flow;
   a carbon dioxide gas input configured to input carbon dioxide into said at least one downcomer tower near a bottom of said downcomer tower and configured to create a carbon dioxide gas upward flow in said downcomer tower;
   a counterflow created with said algae downward fluid flow and said carbon dioxide gas upward flow in said downcomer tower configured to allow optimum reaction between said algae and said carbon dioxide; and
   a collection tank configured to collect mature algae from said downcomer tower.
2. The system as described in clause 1 or any other clause wherein said optimum reaction between said algae and said carbon dioxide comprises an appropriate amount of reaction time in mixing said algae and said carbon dioxide.
3. The system as described in clause 1 or any other clause wherein said at least one downcomer tower comprises at least two downcomer towers.
4. The system as described in clause 3 or any other clause wherein said at least two downcomer towers are each located outside of said upcomer tower.
5. The system as described in clause 1 or any other clause wherein said upcomer tower comprises said spiral impressions configured to provide said enhanced growth mixing spectrum mixing in said upcomer tower.
6. The system as described in clause 1 or any other clause and further comprising a sparger near said carbon dioxide gas input and near a bottom of said downcomer tower and configured to create gas bubbles with said carbon dioxide gas.
7. The system as described in clause 4 or any other clause wherein said gas bubbles comprise a size of gas bubbles chosen from nanobubble size and microbubble size.
8. The system as described in clause 1 or any other clause wherein said fluid having said algae comprises nutrients in said fluid.
9. The system as described in clause 1 or any other clause and further comprising a filter located near a top of said upcomer tower and configured to filter carbon dioxide from said gas.
10. The system as described in clause 9 or any other clause and further comprising a gas release configured to release other gases filtered from said gas, wherein said other gases are chosen from nitrogen, oxygen, trace gases, and any combination thereof.
11. The system as described in clause 1 or any other clause wherein said gas is chosen from carbon dioxide, air, flue gas, and gas from a smokestack.
12. The system as described in clause 9 or any other clause and further comprising a gas circulation component configured to circulate said filtered carbon dioxide to said bottom of said downcomer tower.
13. The system as described in clause 1 or any other clause wherein said counterflow created with said algae downward fluid flow and said carbon dioxide gas upward flow in said downcomer tower is configured consume said carbon dioxide with said reaction between said algae and said carbon dioxide.
14. The system as described in clause 13 or any other clause wherein said counterflow created with said algae downward fluid flow and said carbon dioxide gas upward flow in said downcomer tower is configured consume substantially all of said carbon dioxide with said reaction between said algae and said carbon dioxide.
15. The system as described in clause 1 or any other clause wherein said towers are made of clear tubing.
16. The system as described in clause 9 or any other clause wherein said filter is chosen from a graphene filter, a membrane, molecular filter, and metal oxide framework filter.
17. The system as described in clause 6 or any other clause wherein said sparger comprises a fine mesh disk, a microbubble generator, or a nanobubble generator.
18. The system as described in clause 1 or any other clause and further comprising a downward fluid flow velocity adjuster configured to adjust a velocity of said downward fluid flow in said downcomer tower.
19. The system as described in clause 1 or any other clause and further comprising a spiral impression adjuster configured to adjust a depth of said spiral impressions in said tower.
20. The system as described in clause 1 or any other clause and further comprising an algae filter and dewaterer near said bottom of said downcomer tower configured to filter and remove water from mature algae in said downcomer tower and direct said filtered mature algae to said collection tank.
21. The system as described in clause 1 or any other clause and further comprising an algae coagulator near said bottom of said downcomer tower configured to coagulate mature algae in said downcomer tower.
22. The system as described in clause 21 or any other clause wherein said algae coagulator comprises a charge plate.
23. The system as described in clause 20 or any other clause wherein said algae filter and dewaterer comprises a Coanda screen.
24. The system as described in clause 1 or any other clause and further comprising a high curvature flow of said fluid flow in said downcomer configured to filter out heavier algae from said fluid flow.
25. The system as described in clause 1 or any other clause wherein said upcomer tower connected to said at least one downcomer tower creates a single photobioreactor system.
26. The system as described in clause 25 or any other clause and further comprising a plurality of single photobioreactor systems connected together.
27. The system as described in clause 26 or any other clause wherein said plurality of single photobioreactor systems connected together each utilize a single algae-fluid input and a single mature algae output.
28. The system as described in clause 26 or any other clause and further comprising separate operation controls for each of said single photobioreactor systems when connected together.
29. The system as described in clause 28 or any other clause wherein said separate operation controls are automatically controlled in response to sensor inputs from each single photobioreactor system.
30. The system as described in clause 28 or any other clause wherein said separate operation controls comprises control of valves associated in each of said single photobioreactor system and configured to open and close inputs and outputs for each single photobioreactor system.
31. The system as described in clause 25 or any other clause and further comprising a horizontal piping section having said spiral impressions configured to provide said enhanced growth mixing spectrum mixing connected as part of said single photobioreactor system and configured to grow algae in said horizontal piping system.
32. The system as described in clause 1 or any other clause and further comprising an integrated ethanol-corn-fertilizer-algae system configured to integrate said photobioreactor system with an industry chosen from a fertilizer plant, corn fields, ethanol plant, and any combination thereof.
33. The system as described in clause 32 or any other clause wherein said integrated ethanol-corn-fertilizer-algae system is configured to supply at least one byproduct chosen from carbon dioxide, ammonium runoff, stillage, and waste heat to said photobioreactor system.
34. The system as described in clause 32 or any other clause wherein said integrated ethanol-corn-fertilizer-algae system is configured to produce at least one product from said photobioreactor system chosen from oil, nutraceuticals, organic fertilizer, astaxanthin, protein, and phycocyanin.
35. The system as described in clause 1 or any other clause and further comprising an integrated coal or natural gas-wastewater-algae system configured to integrate said photobioreactor system with an industry chosen from a wastewater treatment plant, a coal powerplant, and a natural gas powerplant.
36. The system as described in clause 35 or any other clause wherein said integrated coal or natural gas-wastewater-algae system is configured to supply at least one byproduct chosen from effluent, carbon dioxide, electricity, and wastewater to said photobioreactor system.
37. The system as described in clause 36 or any other clause wherein said integrated coal or natural gas-wastewater-algae system is configured to produce at least one product from said photobioreactor system chosen from oil, nutraceuticals, organic fertilizer, astaxanthin, protein, and phycocyanin.
38. A method for using a photobioreactor comprising the steps of:
   providing at least one downcomer tower having spiral impressions;
   creating an enhanced growth mixing spectrum mixing in said downcomer tower with said spiral impressions;
   connecting an upcomer tower to said at least one downcomer tower;
   inputting a fluid having algae into said at least one downcomer tower near a top of said downcomer tower with a fluid input;
   creating an algae downward fluid flow in said downcomer tower;
   inputting a gas into said upcomer tower near a bottom of said upcomer tower with a gas input;
   driving a system flow with said gas;
   creating a gas upward flow in said upcomer tower;
   inputting carbon dioxide into said at least one downcomer tower near a bottom of said downcomer tower with a carbon dioxide input;
   creating a carbon dioxide gas upward flow in said downcomer tower;
   creating a counterflow with said algae downward fluid flow and said carbon dioxide gas upward flow in said downcomer tower;
   optimally reacting said algae and said carbon dioxide in said counterflow; and collecting mature algae from said downcomer tower in a collection tank.
39. The method as described in clause 38 or any other clause wherein said optimally reacting said algae and said carbon dioxide in said counterflow comprises an appropriate amount of reaction time in mixing said algae and said carbon dioxide.
40. The method as described in clause 38 or any other clause wherein said at least one downcomer tower comprises at least two downcomer towers.
41. The method as described in clause 40 or any other clause wherein said at least two downcomer towers are each located outside of said upcomer tower.
42. The method as described in clause 38 or any other clause and further comprising a step of providing said enhanced growth mixing spectrum mixing in said upcomer tower with spiral impressions in said upcomer tower.
43. The method as described in clause 38 or any other clause and further comprising a step of creating gas bubbles with said carbon dioxide gas with a sparger located near said carbon dioxide gas input.
44. The method as described in clause 43 or any other clause wherein said gas bubbles comprise a size of gas bubbles chosen from nanobubble size and microbubble size.
45. The method as described in clause 38 or any other clause wherein said fluid comprises nutrients.
46. The method as described in clause 45 or any other clause and further comprising filtering carbon dioxide from said gas with a filter located near said top of said upcomer tower.
47. The system as described in clause 46 or any other clause and further comprising releasing other gases filtered from said gas with a gas release, wherein said other gases are chosen from nitrogen, oxygen, trace gases, and any combination thereof.
48. The method as described in clause 38 or any other clause wherein said gas is chosen from carbon dioxide, air, flue gas, and gas from a smokestack.
49. The method as described in clause 46 or any other clause and further comprising circulating said filtered carbon dioxide to said bottom of said downcomer tower with a gas circulation component.
50. The method as described in clause 38 or any other clause and further comprising consuming said carbon dioxide with said reaction between said algae and said carbon dioxide in said counterflow.
51. The method as described in clause 50 or any other clause and further comprising consuming substantially all of said carbon dioxide with said reaction between said algae and said carbon dioxide in said counterflow.
52. The method as described in clause 38 or any other clause wherein said towers are made of clear tubing.
53. The method as described in clause 46 or any other clause wherein said filter is chosen from a graphene filter, a membrane, molecular filter, and metal oxide framework filter.
54. The method as described in clause 43 or any other clause wherein said sparger comprises a fine mesh disk, a microbubble generator, or a nanobubble generator.
55. The method as described in clause 38 or any other clause and further comprising adjusting a velocity of said downward fluid flow in said downcomer tower with a downward fluid flow velocity adjuster.
56. The method as described in clause 38 or any other clause and further comprising adjusting a depth of said spiral impressions in said tower.
57. The method as described in clause 38 or any other clause and further comprising filtering and dewatering mature algae in said downcomer tower with an algae filter and dewaterer near said bottom of said downcomer tower and directing said filtered mature algae to said collection tank.
58. The method as described in clause 38 or any other clause and further comprising coagulating mature algae in said downcomer tower with an algae coagulator near said bottom of said downcomer tower.
59. The method as described in clause 58 or any other clause wherein said algae coagulator comprises a charge plate.
60. The method as described in clause 57 or any other clause wherein said algae filter and dewaterer comprises a Coanda screen.
61. The method as described in clause 38 or any other clause and further comprising a high curvature flow of said fluid flow in said downcomer configured to filter out heavier algae from said fluid flow.
62. The method as described in clause 38 or any other clause wherein said upcomer tower connected to said at least one downcomer tower creates a single photobioreactor system.
63. The method as described in clause 62 or any other clause and further comprising connecting a plurality of single photobioreactor systems together.
64. The method as described in clause 63 or any other clause wherein said connected plurality of single photobioreactor systems each utilize a single algae-fluid input and a single mature algae output.
65. The method as described in clause 63 or any other clause and further comprising separate operation controls for each of said single photobioreactor systems when connected together.
66. The method as described in clause 65 or any other clause wherein said separate operation controls are automatically controlled in response to sensor inputs from each single photobioreactor system.
67. The method as described in clause 65 or any other clause wherein said separate operation controls control valves associated in each of said single photobioreactor system and control opening and closing inputs and outputs for each single photobioreactor system.
68. The method as described in clause 62 or any other clause and further comprising providing a horizontal piping section having said spiral impressions to create said enhanced growth mixing spectrum mixing; connecting said horizontal piping system as part of said single photobioreactor system; and growing algae in said horizontal piping system.
69. The method as described in clause 38 or any other clause and further comprising integrating said photobioreactor system with an industry chosen from a fertilizer plant, corn fields, ethanol plant, and any combination thereof.
70. The method as described in clause 69 or any other clause and further comprising supplying at least one byproduct from said industry chosen from carbon dioxide, ammonium runoff, stillage, and waste heat to said photobioreactor system.
71. The method as described in clause 69 or any other clause and further comprising producing at least one product from said photobioreactor system chosen from oil, nutraceuticals, organic fertilizer, astaxanthin, protein, and phycocyanin.
72. The method as described in clause 38 or any other clause and further comprising integrating said photobioreactor system with an industry chosen from a wastewater treatment plant, a coal powerplant, and a natural gas powerplant.
73. The method as described in clause 72 or any other clause and further comprising supplying at least one byproduct chosen from effluent, carbon dioxide, electricity, and wastewater from said industry to said photobioreactor system.
74. The method as described in clause 72 or any other clause and further comprising producing at least one product from said photobioreactor system chosen from oil, nutraceuticals, organic fertilizer, astaxanthin, protein, and phycocyanin.
75. The method as described in clause 57 or any other clause and further comprising a step of circulating said fluid flow after said mature algae has been filtered to a top of a downcomer.
76. The method as described in clause 75 or any other clause wherein said circulated fluid flow comprises young algae, nutrients, fluid, and any combination thereof.

As can be easily understood from the foregoing, the basic concepts of the various embodiments of the present invention(s) may be embodied in a variety of ways. It involves both bioreactor techniques as well as devices to accomplish the appropriate bioreactor. In this application, the bioreactor techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the various embodiments of the invention(s) and may not explicitly show how each feature or element can actually be representative of a broader function or of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc., as well as terms that encompass largely but not wholly that which is specified. Further, for this application if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified). In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person of ordinary skill in this field. Where the application is described in device-oriented terminology, each element of the device implicitly performs a function. Apparatus claims may not only be included for the device described, but also method or process claims may be included to address the functions of the embodiments and that each element performs. Neither the description nor the terminology is intended to limit the scope of the claims that will be included in any subsequent patent application.

It should also be understood that a variety of changes may be made without departing from the essence of the various embodiments of the invention(s). Such changes are also implicitly included in the description. They still fall within the scope of the various embodiments of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of embodiments of the invention(s) both independently and as an overall system.

Further, each of the various elements of the embodiments of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the various embodiments of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments of the invention(s) is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “flow” should be understood to encompass disclosure of the act of “flowing”—whether explicitly discussed or not—and, conversely, were there effectively disclosure of the act of “flowing”, such a disclosure should be understood to encompass disclosure of a “flow” and even a “means for flowing.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components, programming, subroutines, logic, or elements that are configured to, or configured and arranged to, provide or even achieve a particular result, use, purpose, situation, function, or operation, or as components that are capable of achieving a particular activity, result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of reference below or any other information statement filed with the application are hereby appended and hereby incorporated by reference, however, as to each of the above, to the extent that such information or statements incorporated by reference might he considered inconsistent with the patenting of the various embodiments of invention(s) such statements are expressly not to be considered as made by the applicant(s).

### U.S. Patents

| | Kind | | Name of Patentee or Applicant |
|---|---|---|---|
| Pat. No. | Code | Issue Date | of cited Document |
| 8715980 | B2 | 2014 May 6 | Clarke |
| 11028355 | B2 | 2021 Jun. 8 | Falco |
| 7824904 | B1 | 2010 Nov. 2 | Dimanshteyn |
| 5846816 | | 1998 Dec. 8 | Forth |
| 8734805 | B2 | 2014 May 27 | Hentges et al. |
| 9295206 | B2 | 2016 Mar. 29 | Jovine |
| 8507264 | B2 | 2013 Aug. 13 | Lewnard et al. |
| 9102923 | B2 | 2015 Aug. 11 | Meiser et al. |
| 3496605 | | 1970 Feb. 24 | Onaka |
| 8409845 | B2 | 2013 Apr. 2 | Trent et al. |
| 2732663 | | 1956 Jan. 31 | Dewey |
| 4473970 | | 1984 Oct. 2 | Hills |
| 4233958 | | 1980 Nov. 18 | Heden |
| 4868123 | | 1989 Sep. 19 | Berson et al. |
| 6827036 | | 2004 Dec. 7 | Connolly |
| 3650068 | | 1972 Mar. 21 | Meyer et al. |
| 3468057 | | 1969 Sep. 23 | Buisson et al. |
| 4217728 | | 1980 Aug. 19 | Shimamatsu et al. |
| 7682821 | B2 | 2010 Mar. 23 | Woods et al. |
| 9938492 | B2 | 2018 Apr. 10 | Gressel et al. |

### U.S. Patent Application Publications

| | | | Name of Patentee |
|---|---|---|---|
| Publication | Kind | Publication | or Applicant |
| Number | Code | Date | of cited Document |
| 20200369995 | A1 | 2020 Nov. 26 | Falco |
| 20140329297 | | 2014 Nov. 6 | Longan |
| 20100068801 | | 2012 Dec. 4 | Woods |
| 20080316858 | A1 | 2008 Dec. 25 | Gron |
| 20090130706 | A1 | 2009 May 21 | Berzin et al. |
| 20140186909 | A1 | 2014 Jul. 3 | Calzia et al. |
| 20160369216 | A1 | 2016 Dec. 22 | Matsumoto |
| 20140315290 | A1 | 2014 Oct. 23 | Mottahedeh |
| 20140134672 | A1 | 2014 May 15 | Tuttman et al. |
| 20080153080 | A1 | 2008 Jun. 26 | Woods et al. |
| 20130109085 | A1 | 2013 May 2 | Woods et al. |
| 20050260553 | A1 | 2005 Nov. 24 | Berzin |
| 20180223241 | A1 | 2018 Aug. 9 | Zhongzhi He |
| 20180119083 | A1 | 2018 May 3 | Zheng et al. |

### Foreign Patent Application Publications

| Foreign | | | | |
|---|---|---|---|---|
| Document | Country | Kind | Publication | Name of Patentee or Applicant |
| Number | Code | Code | Date | of cited Document |
| 2020/237103 | WO | A1 | 2020 Nov. 26 | SolarClean Fuels, LLC |
| 2016/054939 | WO | A1 | 2016 Apr. 14 | Chi ct al. |
| 2479340 | ES | A2 | 2015 Jul. 7 | Gomez et al. |
| 102134553 | CN | A | 2011 Jul. 27 | Fei et al. |
| 2004074423 | WO | A2 | 2004 Sep. 2 | Tredici et al. |
| 102134553 | CN | A | 2013 Nov. 6 | Institute of Process Engineering |
| 2020237103 | WO | A1 | 2020 Nov. 26 | SolarClean Fuels, LLC |
| 2020277475 | AU | | 2022 Nov. 3 | SolarClean Fuels, LLC |
| 112021023423-3 | BR | | 2022 Jan. 2 | SolarClean Fuels, LLC |
| 3141655 | CA | | 2023 Jan. 3 | SolarClean Fuels, LLC |
| 114207104 | CN | | 2023 Apr. 25 | SolarClean Fuels, LLC |
| 104263646 | CN | | 2015 Jan. 7 | SDIC |
| 101748054 | CN | A | 2010 Jun. 23 | ENN Science and Technology |
| | | | | Development Co Ltd |
| 102018116698 | DE | A1 | 2019 Jan. 24 | Holzrichter |
| 202117055375 | IN | A | 2022 Mar. 15 | SolarClean Fuels, LLC |
| 7127913 | JP | | 2022 Aug. 22 | SolarClean Fuels, LLC |
| 397542 | MX | | 2022 Nov. 17 | SolarClean Fuels, LLC |

### Non-Patent Literature Documents

Putt, et al. An efficient system for carbonation of high-rate algae pond water to enhance
CO2 mass transfer. Bioresource Technology 102 (2011) 2340-3245.
Wiley, Patrick Microalgae Cultivation using Offshore Membrane Enclosures for Growing
Algae (OMEGA). 2013. 95 pages.
Batchelor, G. K., 1967 An Introduction to Fluid Dynamics, Cambridge University Press,
London.
Kwak et al 2020, Experimental study of nanoparticle transport and penetration efficiency
on a sharp-bent tube (elbow connection), International Journal of Heat and Mass Transfer,
155, 119816.
Kwak et al, 2021, Numerical investigation of nanoparticle deposition location and pattern
on a sharp-bent tube wall, International Journal of Heat and Mass Transfer, 164, 120534.
Ying, et al 2013, Enhanced Mass Transfer in Microbubble Driven Airlift Bioreactor for
Microalgal Culture”, Engineering, 5, 735-743.
Wu, et al 2021, Optimizing the growth of Haematococcus pluvialis based on a novel
microbubble-driven photobioreactor, iScience 24, 103461.
Zimmerman, et al 2011, Design of an airlift loop bioreactor and pilot scales studies with
fluidic oscillator induced microbubbles for growth of a microalgae Dunaliella salina,
Applied Energy, 88 3357-3369.
Promthaisong, Pitak, et al., Numerical investigation on turbulent forced convection and heat
transfer characteristic in spirally semicircle-grooved tube. International Journal of
Mechanical and Materials Engineering, (2016) 11: 9 DOI 10.1186/s40712-016-0062-2. 15 pages.
PERRINE, et al., Optimization of photosynthetic light energy utilization by microalgae,
Algal Research 1 (2012) 134-142, Accepted 4 Jul. 2012, 9 pages
ADVANCED ALGAL SYSTEMS TECHNOLOGY AREA, 2017 Project Peer Review, 110 pages
AKHAVAN-BEHABADI, et al., Pressure drop and heat transfer augmentation due to
coiled wire inserts during laminar flow of oil inside a horizontal tube, International Journal
of Thermal Sciences 49 (2010) 373-379, Accepted 14 Jun. 2009, 7 pages
DE GODOS, et al., Evaluation of carbon dioxide mass transfer in raceway reactors for
microalgae culture using flue gases, Bioresource Technology 1
XI GAO, et al., Comprehensive computational model for combining fluid hydrodynamics,
light transport and biomass growth in a Taylor vortex algal photobioreactor: Eulerian
approach, Algal Research 24 (2017) 1-8, Accepted 16 Mar. 2017, 8 pages
GUNES, et al., Heat transfer enhancement in a tube with equilateral triangle cross sectioned
coiled wire inserts, Experimental Thermal and Fluid Science 34 (2010) 684-691, Accepted
28 Dec. 2009, 8 pages
GUNES, et al., A Taguchi approach for optimization of design parameters in a tube with
coiled wire inserts, Applied Thermal Engineering 31 (2011) 2568e2577, Accepted 12 Apr.
2011, 12 pages
MOOIJ, et al., Impact of light color on photobioreactor productivity, Algal Research 15
(2016) 32-42, Accepted 23 Jan. 2016, 11 pages
KONG, et al., Light-Limited Continuous Culture of Chlorella vulgaris in a Taylor Vortex
Reactor, Environmental Progress & Sustainable Energy (Vol. 32, No. 4) DOI 10.1002/ep,
Aug. 1, 2013
KONG, et al., Enhanced Algal Growth Rate in a Taylor Vortex Reactor, Biotechnology and
Bioengineering, Vol. 110, No. 8, August, 2013, 10 pages
LAWS, et al., Photosynthetic Efficiency Optimization Studies with the Macroalga
Gracilaria tikvihae: Implications for CO2 Emission Control from Power Plants, Bioresource
Technology 37 (1991) 25-33, 9 pages
LIU et al., A comprehensive review on passive heat transfer enhancements in pipe
exchangers, Renewable and Sustainable Energy Reviews 19 (2013) 64-81, Accepted 5
Nov. 2012, 8 pages
The National Alliance for Advanced Biofuels and Bioproducts (NAABB), Full Final
Report Section I, Program Overview, 75 pages. Date Unknown.
The National Alliance for Advanced Biofuels and Bioproducts (NAABB), Full Final
Report Section II, Team R&D Overview, 198 pages. Date Unknown.
The National Alliance for Advanced Biofuels and Bioproducts (NAABB), Full Final
Report Section III, Individual Project Summaries Team R&D, Overview, 139 pages. Date
Unknown.
OLIVARES 2016 National Alliance for Advanced Biofuels and Bioproducts Synopsis
(NAABB), 28 pages
FUENTES et al., Outdoor continuous culture of Porphyridium cruentum in a tubular
photobioreactor: quantitative analysis of the daily cyclic variation of culture parameters,
Journal of Biotechnology 70 (1999) 271-288, accepted 22 Dec. 1998, 18 pages
PROMVONGE, Thermal enhancement in a round tube with snail entry and coiled-wire
inserts, International Communications in Heat and Mass Transfer 35 (2008) 623-629, 31
Jan. 2008, 7 pages
UNKEFER et al., Review of the algal biology program within the National Alliance for
Advanced Biofuels and Bioproducts, Algal Research 22 (2017) 187-215, Accepted 1 Jun.
2016, 29 pages
SAYRE, Microalgae: The Potential for Carbon Capture, BioScience • October 2010/Vol.
60 No. 9, www.biosciencemag.org, 6 pages
SHEEHAN et al., A Look Back at the U.S. Department of Energy's Aquatic Species
Program-Biodiesel from Algae, National Renewable Energy Laboratory NREL/TP-580-
24190, July 1998, 328 pages
TREDICI, Mario R Tredici (2010) Photobiology of microalgae mass cultures:
understanding the tools for the next green revolution, Biofuels, 1:1, 143-162, DOI:
10.4155/bfs.09.10, 21 pages
COWARD et al., Utilising light-emitting diodes of specific narrow wavelengths for the
optimization and co-production of multiple high-value compounds in Porphyridium
purpureum, Bioresource Technology 221 (2016) 607-615, Accepted 22 Sep. 2016, 9 pages
VONSHAK et al., Mass Production of the Blue-green Alga Spirulina: An Overview,
Biomass 15 (1988) 233-247, accepted 28 Jan. 1988, 15 pages
VONSHAK et al., The effect of light availability on the photosynthetic activity and
productivity of outdoor cultures of Arthrospira platensis (Spirulina ), J Appl Phycol (2014)
26: 1309-1315 DOI 10.1007/s10811-013-0133-1, Revised and accepted: 22 Aug. 2013, 7 pages
BABCOCK et al., Hydrodynamics and mass transfer in a tubular airlift photobioreactor, J
Appl Phycol, 14: 169-184, 2002, 16 pages
WAGNER and VOGELMANN, Cultivation of Plant Tissue Cultures in Bioreactors and
Formation of Secondary Metabolites. Plant Tissue Culture and Its Bio-Technological
Application pp. 245-252. Apr. 10, 2019.
GRIMA et al., Scale-up of tubular photobioreactors. J. Appl. Phycol. 12, 355-368, 2000.
PULZ, Photobioreactors: production systems for phototrophic microorganisms. Appl
Microbiol Biotechnol 2001; 57: 287-93, 7 pages
HOSSEINVERDI and FASEL, Laminar-turbulent transition in a laminar separation bubble
in the presence of free-stream turbulence. (c) 2015/Procedia IUTAM 14 (2015) 570-579,
2015, 10 pages
VONSHAK, Outdoor mass production of Spirulina: the basic concept. In: Vonshak A (ed)
Spirulina platensis (Arthrospira): physiology, cell-biology and biotechnology. Taylor and
Francis, Bristol, 1997, pp 79-99, 22 pages
WEISSMAN, et al., Photobioreactor design: mixing, carbon utilization, and oxygen
accumulation, 1988, Biotechnology and Bioengineering, Vol. 31, pp. 336-344.
ALMUTAIRI, et al., Dynamics of laminar separation bubble over NACA-0012 airfoil near
stall conditions. Aerospace Science and Technology 68 (2017) 193-203. Accepted
May 9, 2017, available online May 15, 2017, 11 pages
LEI, et al., Re-examination of the effect of a plane boundary on force and vortex shedding
of a circular cylinder. Journal of Wind Engineering and Industrial Aerdynamics 80 (1999)
263-286, Aug. 31, 1998, 24 pages
HETSCH and RIST, An analysis of the structure of laminar separation bubbles in swept
infinite geometries, European Journal of Mechanics B/Fluids 28 (2009) 486-493, Mar.
12, 2009, 8 pages
TREDICI, M. R. (2004) Mass production of Microalgae: Photobioreactors. Handbook of
Microalgal Culture: Biotechnology and Applied Phycology. Edited by Amos Richmond,
178-214, 37 pages
OGBANNA et al, Industrial-size photobioreactor s. Chemtech: 27(7), p 43-49, 1997, 7 pages
TULECKE and NICKELL, Production of Large Amounts of Plant Tissue by Submerged
Culture. Science, New Series, Vol. 130, No. 3379 (Oct. 2, 1959) 863-864, 3 pages
GUDIN and CHAUMONT, Cell fragility - The Key Problem of Microalgae Mass Production
in closed Photobioreactors, Bioresource Technology 38: 145-151, 1991, 7 pages
Photobioreactor Design for Photobiological Production of Hydrogen. J. C. Ogbonna and H.
Tanaka. Institute of Applied Biochemistry, University of Tsukuba, Tsukuba, Japan. 245-
261. Date unknown.
Lee et al., High-Density Algal Photobioreactors Using Light-Emitting Diodes. Dept of
Chem Eng., University of Michigan. Received Dec. 23, 1993, Accepted Jun. 17, 1994.
Biotechnology and Bioengineering, Vol. 44, Pp. 1161-1167 (1994).
Javanmardian, Minoo, Ph.D., High-density algal cultures: Photobioreactors design and cell
cycle kinetics. The University of Michigan, 1991. 231 pages.
O. Pulz and K. Scheibenbogen, Photobioreactors: Design and Performance with Respect to
Light Energy Input, Advances in Biochemical Engineering/Biotechnology, Vol. 59. 1998.
30 pages.
Miron, et al., Comparative evaluation of compact photobioreactors for large-scale
monoculture of microalgae. Journal of Biotechnology 70 (1999) 249-270.
International PCT Application No. PCT/US20/034097, filed May 21, 2020. First Named
Inventor: Falco. International Search Report dated Aug. 25, 2020. 2 pages.
International PCT Application No. PCT/US20/034097, filed May 21, 2020. First Named
Inventor: Falco. Written Opinion of the International Searching Authority dated Aug. 25, 2020.
10 pages.
Gomez-Perez., et al., Twisted tubular photobioreactor fluid dynamics evaluation for energy
consumption minimization. Algal Research 27 (2017) 65-72.
Effect of pitch on light/dark cycle characteristics of microalgae photo-bioreactor with spiral
fins, Lei Yuling et al., Biotechnology & Business, No. 2, pp. 81-88. Date unknown.

Thus, the applicant(s) should be understood to have support to claim and make claims to embodiments including at least: i) each of the bioreactor devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, it should be understood that in characterizing these and all other aspects of the various embodiments of the invention(s)—whether characterized as a device, a capability, an element, or otherwise, because all of these can be implemented via software, hardware, or even firmware structures as set up for a general purpose computer, a programmed chip or chipset, an ASIC, application specific controller, subroutine, logic, or other known programmable or circuit specific structure—it should be understood that all such aspects are at least defined by structures including, as person of ordinary skill in the art would well recognize: hardware circuitry, firmware, programmed application specific components, and even a general purpose computer programmed to accomplish the identified aspect. For such items implemented by programmable features, the applicant(s) should be understood to have support to claim and make a statement of invention to at least: xv) processes performed with the aid of or on a computer, machine, or computing machine as described throughout the above discussion, xvi) a programmable apparatus as described throughout the above discussion, xvii) a computer readable memory encoded with data to direct a computer comprising means or elements which function as described throughout the above discussion, xviii) a computer, machine, or computing machine configured as herein disclosed and described, xix) individual or combined subroutines, processor logic, and/or programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) a processor executing instructions that act to achieve the steps and activities detailed, xxvi) circuitry configurations (including configurations of transistors, gates, and the like) that act to sequence and/or cause actions as detailed, xxvii) computer readable medium(s) storing instructions to execute the steps and cause activities detailed, xxviii) the related methods disclosed and described, xxix) similar, equivalent, and even implicit variations of each of these systems and methods, xxx) those alternative designs which accomplish each of the functions shown as are disclosed and described, xxxi) those alternative designs and methods which accomplish each of the functions shown as are implicit to accomplish that which is disclosed and described, xxxii) each feature, component, and step shown as separate and independent inventions, and xxxiii) the various combinations of each of the above and of any aspect, all without limiting other aspects in addition.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrases “comprising”, “including”, “containing”, “characterized by” and “having” are used to maintain the “open-end” claims herein, according to traditional claim interpretation including that discussed in MPEP § 2111.03. Thus, unless the context requires otherwise, it should be understood that the terms “comprise” or variations such as “comprises” or “comprising”, “include” or variations such as “includes” or “including”, “contain” or variations such as “contains” and “containing”, “characterized by” or variations such as “characterizing by”, “have” or variations such as “has” or “having”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 9 or any other claim” or the like, it could be re-drafted as dependent on claim 1, claim 8, or even claim 11 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A photobioreactor system comprising:
at least two downcomer towers, each having spiral impressions configured to provide an enhanced growth mixing spectrum mixing in each of said at least two downcomer towers;
an upcomer tower connected to said at least two downcomer towers;
an algae-fluid input configured to input a fluid having algae into each of said at least two downcomer towers, wherein said algae-fluid input is located near a top of each of said at least two downcomer towers;
a gas input configured to input gas into said upcomer tower near a bottom of said upcomer tower, configured to create a gas upward flow in said upcomer tower, and configured to create an algae downward fluid flow in each of said two downcomer towers, wherein said gas comprises air;
a carbon dioxide gas input configured to input carbon dioxide gas into each of said two downcomer towers near a bottom of each of said downcomer tower and configured to create a carbon dioxide gas upward flow in each of said downcomer towers;
a counterflow created with said algae downward fluid flow and said carbon dioxide gas upward flow in each of said at least two downcomer towers configured to allow optimum reaction between said algae and said carbon dioxide so that substantially all of said carbon dioxide gas inputted by said carbon dioxide gas input is consumed by said algae; and
a collection tank configured to collect mature algae from each of said two downcomer towers.

2. The system as described in claim 1 wherein said optimum reaction between said algae and said carbon dioxide comprises an appropriate amount of reaction time in mixing said algae and said carbon dioxide.

3. (canceled)

4. The system as described in claim 1 wherein said at least two downcomer towers are each located outside of said upcomer tower.

5. The system as described in claim 1 wherein said upcomer tower comprises said spiral impressions configured to provide said enhanced growth mixing spectrum mixing in said upcomer tower.

6. The system as described in claim 1 and further comprising a sparger near said carbon dioxide gas input and near a bottom of said downcomer tower and configured to create gas bubbles with said carbon dioxide gas.

7-8. (canceled)

9. The system as described in claim 1 and further comprising a filter located near a top of said upcomer tower and configured to filter carbon dioxide from a gas source.

10. The system as described in claim 9 and further comprising a gas release configured to release other gases filtered from said gas source, wherein said other gases are chosen from nitrogen, oxygen, trace gases, and any combination thereof.

11. The system as described in claim 9 wherein said gas source is chosen from carbon dioxide, air, flue gas, and gas from a smokestack.

12. The system as described in claim 9 and further comprising a gas circulation component configured to circulate said filtered carbon dioxide to said bottom of said downcomer tower.

13-14. (canceled)

15. The system as described in claim 1 wherein said towers are made of clear tubing.

16-17. (canceled)

18. The system as described in claim 1 and further comprising a downward fluid flow velocity adjuster configured to adjust a velocity of said algae downward fluid flow in each of said at least two downcomer towers.

19. The system as described in claim 1 and further comprising a spiral impression adjuster configured to adjust a depth of said spiral impressions in said tower.

20. The system as described in claim 1 and further comprising an algae filter and dewaterer near said bottom of said downcomer tower configured to filter and remove water from mature algae in said downcomer tower and direct said filtered mature algae to said collection tank.

21. The system as described in claim 1 and further comprising an algae coagulator near said bottom of said downcomer tower configured to coagulate mature algae in said downcomer tower.

22-23. (canceled)

24. The system as described in claim 1 and further comprising a high curvature flow of said fluid flow in said downcomer tower configured to direct said fluid flow around a bend near a bottom of said at least two downcomer towers, allow mature heavy algae to fall below said fluid flow, and to filter out said mature heavy algae from said fluid flow.

25. The system as described in claim 1 wherein said upcomer tower connected to said at least two downcomer towers creates a single photobioreactor system.

26. The system as described in claim 25 and further comprising a plurality of single photobioreactor systems connected together.

27-30. (canceled)

31. The system as described in claim 25 and further comprising a horizontal piping section having said spiral impressions configured to provide said enhanced growth mixing spectrum mixing connected as part of said single photobioreactor system and configured to grow algae in said horizontal piping system.

32. The system as described in claim 1 and further comprising an integrated ethanol-corn-fertilizer-algae system configured to integrate said photobioreactor system with an industry chosen from a fertilizer plant, corn fields, ethanol plant, and any combination thereof.

33. The system as described in claim 32 wherein said integrated ethanol-corn-fertilizer-algae system is configured to supply at least one byproduct chosen from carbon dioxide, ammonium runoff, stillage, and waste heat to said photobioreactor system.

34. The system as described in claim 32 wherein said integrated ethanol-corn-fertilizer-algae system is configured to produce at least one product from said photobioreactor system chosen from oil, nutraceuticals, organic fertilizer, astaxanthin, protein, and phycocyanin.

35. The system as described in claim 1 and further comprising an integrated coal or natural gas-wastewater-algae system configured to integrate said photobioreactor system with an industry chosen from a wastewater treatment plant, a coal powerplant, and a natural gas powerplant.

36. The system as described in claim 35 wherein said integrated coal or natural gas-wastewater-algae system is configured to supply at least one byproduct chosen from effluent, carbon dioxide, electricity, and wastewater to said photobioreactor system.

37. The system as described in claim 36 wherein said integrated coal or natural gas-wastewater-algae system is configured to produce at least one product from said photobioreactor system chosen from oil, nutraceuticals, organic fertilizer, astaxanthin, protein, and phycocyanin.

38. A method for using a photobioreactor comprising the steps of:
providing at least two downcomer towers each having spiral impressions;
creating an enhanced growth mixing spectrum mixing in each of said at least two downcomer towers with said spiral impressions;
connecting an upcomer tower to said at least two downcomer towers;
inputting a fluid having algae into said at least two downcomer towers with a fluid input;
inputting a gas into said upcomer tower near a bottom of said upcomer tower with a gas input, wherein said gas comprises air;
driving a system flow with said gas;
creating a gas upward flow in said upcomer tower;
creating an algae downward fluid flow in each of said two downcomer towers with said inputted gas and said gas upward flow in said upcomer tower;
inputting carbon dioxide gas into each of said two downcomer towers with a carbon dioxide input, wherein said carbon dioxide input is located near a bottom of each of said two downcomer towers;
creating a carbon dioxide gas upward flow in each of said two downcomer towers;
creating a counterflow with said algae downward fluid flow and said carbon dioxide gas upward flow in each of said two downcomer towers;
optimally reacting said algae and said carbon dioxide in said counterflow so that substantially all of said inputted carbon dioxide is consumed by said algae; and
collecting mature algae from each of said at least two downcomer towers in a collection tank.

39. The method as described in claim 38 wherein said optimally reacting said algae and said carbon dioxide in said counterflow comprises an appropriate amount of reaction time in mixing said algae and said carbon dioxide.

40. (canceled)

41. The method as described in claim 38 wherein said at least two downcomer towers are each located outside of said upcomer tower.

42. The method as described in claim 38 and further comprising a step of providing said enhanced growth mixing spectrum mixing in said upcomer tower with spiral impressions in said upcomer tower.

43-47. (canceled)

48. The method as described in claim 38 wherein said gas comprises additional gases chosen from nitrogen and carbon dioxide.

49-51. (canceled)

52. The method as described in claim 38 wherein said towers are made of clear tubing.

53-54. (canceled)

55. The method as described in claim 38 and further comprising adjusting a velocity of said downward fluid flow in each of said at least two downcomer towers with a downward fluid flow velocity adjuster.

56. The method as described in claim 38 and further comprising adjusting a depth of said spiral impressions in said tower.

57. The method as described in claim 38 and further comprising filtering and dewatering mature algae in each of said at least two downcomer towers with an algae filter and dewaterer near said bottom of said downcomer tower and directing said filtered mature algae to said collection tank.

58. The method as described in claim 38 and further comprising coagulating mature algae in said downcomer tower with an algae coagulator near said bottom of at least one of said at least two downcomer towers.

59-60. (canceled)

61. The method as described in claim 38 and further comprising:
providing a high curvature flow of said fluid flow near a bottom of each of said at least two downcomer towers;
directing said fluid flow around a bend in each of said two downcomer towers;
allowing mature heavy algae to fall below said fluid flow; and
collecting said mature heavy algae near said bottom of said downcomer tower.

62. The method as described in claim 38 wherein said upcomer tower connected to said at least two downcomer towers creates a single photobioreactor system.

63. The method as described in claim 62 and further comprising connecting a plurality of single photobioreactor systems together.

64-67. (canceled)

68. The method as described in claim 62 and further comprising providing a horizontal piping section having said spiral impressions to create said enhanced growth mixing spectrum mixing; connecting said horizontal piping system as part of said single photobioreactor system; and growing algae in said horizontal piping system.

69. The method as described in claim 38 and further comprising integrating said photobioreactor system with an industry chosen from a fertilizer plant, corn fields, ethanol plant, and any combination thereof.

70. The method as described in claim 69 and further comprising supplying at least one byproduct from said industry chosen from carbon dioxide, ammonium runoff, stillage, and waste heat to said photobioreactor system.

71. The method as described in claim 69 and further comprising producing at least one product from said photobioreactor system chosen from oil, nutraceuticals, organic fertilizer, astaxanthin, protein, and phycocyanin.

72. The method as described in claim 38 and further comprising integrating said photobioreactor system with an industry chosen from a wastewater treatment plant, a coal powerplant, and a natural gas powerplant.

73. The method as described in claim 72 and further comprising supplying at least one byproduct chosen from effluent, carbon dioxide, electricity, and wastewater from said industry to said photobioreactor system.

74. The method as described in claim 72 and further comprising producing at least one product from said photobioreactor system chosen from oil, nutraceuticals, organic fertilizer, astaxanthin, protein, and phycocyanin.

75. The method as described in claim 57 and further comprising a step of circulating said fluid flow after said mature algae has been filtered to a top of said at least one of two downcomer towers.

76-77. (canceled)

78. The system as described in claim 24 wherein said high curvature flow comprises at least a 135-degree angle bend near said bottom of said downcomer tower.

79. (canceled)

80. The system as described in claim 1 wherein said algae-fluid input comprises an algae-fluid input configured to input said fluid having C. merolae algae into each of said two downcomer towers.

81. The system as described in claim 1 wherein a diameter of each of said two downcomer towers and said upcomer tower is greater than about 6 inches or greater than about 6.625 inches.

82-83. (canceled)

84. The method as described in claim 38 wherein said step of inputting said fluid having said algae into said two downcomer towers with said fluid input comprises a step of inputting said fluid having said C. merolae algae into said two downcomer towers with said fluid input.

85. The method as described in claim 38 and further comprising a step of providing a diameter for each of said two downcomer towers and said upcomer tower that is greater than about 6 inches or greater than about 6.625 inches.

86. The system as described in claim 1 wherein said algae and said mature algae comprises mixotrophic algae.

87. The system as described in claim 86 and further comprising a product produced from said mixotrophic algae comprising phycocyanin.

88. The system as described in claim 86 and further comprising controlling a light-dark cycle of said mixotrophic algae in said at least two downcomer towers.

89. The method as described in claim 38 wherein said step of collecting mature algae from each of said at least two downcomer towers in said collection tank comprises a step of collecting mature mixotrophic algae from each of said at least two downcomer towers in a collection tank.

90. The method as described in claim 89 and further comprising a step of producing a product from said mature mixotrophic algae comprising phycocyanin.

91. The method as described in claim 89 and further comprising a step of controlling a light-dark cycle of said mixotrophic algae in said at least two downcomer towers.
